(19) United States
(12) Patent Application Publication
(10) Pub. No.: US 2025/0186741 A1
(43) Pub. Date: Jun. 12, 2025

(54) MEDICAL DEVICE, CATHETER KIT, AND CATHETER CASE

(71) Applicants: CANON KABUSHIKI KAISHA, Tokyo (JP); CANON U.S.A., INC., Melville, NY (US)

(72) Inventors: Yoshimichi IKEDA, Shizuoka (JP); Daisuke KANEKO, Suntou-gun (JP); Ryo IWASAWA, Kawasaki-shi (JP); Charles George HWANG, Wellesley, MA (US)

(21) Appl. No.: 18/842,274

(22) PCT Filed: Feb. 23, 2023
(86) PCT No.: PCT/US2023/013703

§ 371 Date: Aug. 28, 2024

Related U.S. Application Data
(60) Provisional application No. 63/314,763, filed on Feb. 28, 2022.

(51) Int. Cl. A61M 25/01; A61M 25/00

(52) U.S. Cl. CPC A61M 25/0113; A61M 25/002

(57) ABSTRACT

A catheter kit includes a catheter unit and a catheter case. The catheter unit includes a linear member, a frame guiding the linear member, a first bendable portion that bends when a first linear member of the linear member is driven, and an intermediate portion that supports between the first bendable portion and the frame. An accommodation space that accommodates the catheter unit is provided in the catheter case. A restriction portion is provided in the accommodation space, the restriction portion is used to, when the catheter unit and a base unit including a driving source that drives the linear member are coupled to each other, calibrate a coupled state between the linear member and the driving source.

### PRIORITY AND INCORPORATION BY REFERENCE

The present application claims priority to U.S. Provisional Patent Application Ser. No. 63/314,763, filed Feb. 28, 2022 the disclosure of which is hereby incorporated herein by reference in its entirety.

### FIELD OF THE DISCLOSURE

The present disclosure relates to a medical device, and a catheter kit used in the medical device.

### BACKGROUND

PTL 1 describes a medical tool. The medical tool includes a catheter unit having a deformable portion, and a base unit that deforms the deformable portion. In the medical tool, the catheter unit is detachably attachable to the base unit. When the catheter unit is attached to the base unit, a wire of the catheter unit and a coupling portion of the base unit need to be reliably coupled to each other. For this reason, the wire and the coupling portion are configured to be engaged with each other such that the wire and the coupling portion overlap each other in a direction orthogonal to a moving direction (longitudinal direction) of the wire, that is, in a cross-sectional direction.

### CITATION LIST

### Patent Literature

PTL 1 US2021/0121051

In PTL 1, there is an inconvenience that, when the catheter unit is attempted to be attached to the base unit in a state where the catheter unit is bent, there arises a deviation in position between the wire of the catheter unit and the coupling portion of the base unit and, as a result, coupling is difficult.

### SUMMARY OF THE DISCLOSURE

It is an object of the present disclosure to provide a catheter unit that allows a base unit to be easily attached to the catheter unit, and a medical device including the catheter unit.

A catheter kit includes a catheter unit and a catheter case. The catheter unit includes a linear member, a frame guiding the linear member, a first bendable portion that bends when a first linear member of the linear member is driven, and an intermediate portion that supports between the first bendable portion and the frame. An accommodation space that accommodates the catheter unit is provided in the catheter case.

A restriction portion is provided in the accommodation space, the restriction portion is used to, when the catheter unit and a base unit including a driving source that drives the linear member are coupled to each other, calibrate a coupled state between the linear member and the driving source.

As described above, according to the embodiments, by using the catheter kit including the restriction portion used to calibrate the coupled state between the linear member and the driving source, a user is able to easily attach and detach the catheter unit including the plurality of linear members to the base unit. In addition, it is possible to provide a medical device including the catheter kit.

### BRIEF DESCRIPTION OF DRAWINGS

FIG. 1 is an overall view of a medical system.

FIG. 2 is a perspective view that shows a medical device and a support.

FIG. 3A-B are views that illustrates a catheter.

FIG. 4A-B are views that illustrates a catheter unit.

FIG. 5A-C are views that illustrates a base unit and a wire drive portion.

FIG. 6A-C are views that illustrates the wire drive portion, a coupling device, and a bend drive portion.

FIG. 7A-B are views that illustrates attachment of the catheter unit.

FIG. 8A-B are views that illustrates coupling of the catheter unit with the base unit.

FIG. 9 is an exploded view that illustrates coupling of the catheter unit with the base unit.

FIG. 10 is a view that illustrates fixing of a drive wire by a coupling portion.

FIG. 11 is a view that illustrates fixing of the drive wire with the coupling portion.

FIG. 12 is a view that illustrates fixing of the drive wire with the coupling portion.

FIG. 13 is a view that illustrates fixing of the drive wire with the coupling portion.

FIG. 14A-B are views that illustrates fixing of the drive wire with the coupling portion.

FIG. 15 is a view that illustrates fixing of the drive wire by the coupling portion.

FIG. 16 is a view that illustrates fixing of the drive wire by the coupling portion.

FIG. 17 is a view that illustrates fixing of the drive wire by the coupling portion.

FIG. 18 is a view that illustrates fixing of the drive wire by the coupling portion.

FIG. 19A-B are views that illustrates a storage configuration of the catheter unit.

FIG. 20A-C are views that illustrates the storage configuration of the catheter unit.

FIG. 21 is a view that illustrates the storage configuration of the catheter unit.

FIG. 22A-B are views that illustrates the storage configuration of the catheter unit.

FIG. 23 is a view that illustrates the storage configuration of the catheter unit.

FIG. 24A-B are views that illustrates the storage configuration of the catheter unit.

### DETAILED DESCRIPTION

Hereinafter, the configuration of the present disclosure will be illustrated with reference to the drawings. The dimensions, materials, and shapes of components that will be described in the present embodiments, the arrangement of the components, and the like, should be changed as needed depending on the configuration of an apparatus to which the present disclosure is applied, various conditions, or the like.

### First Embodiment

### <Medical System and Medical Device>

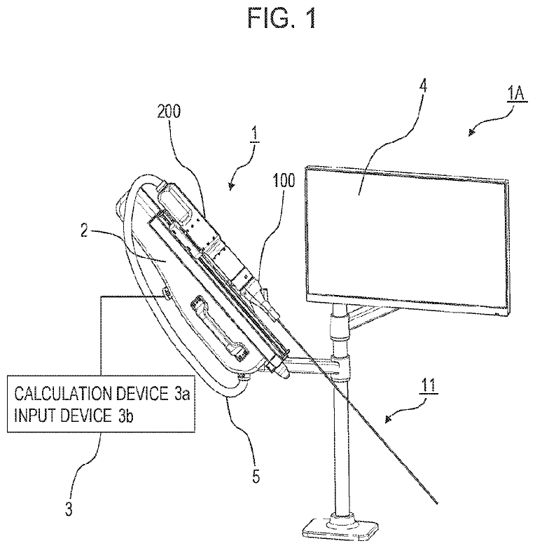

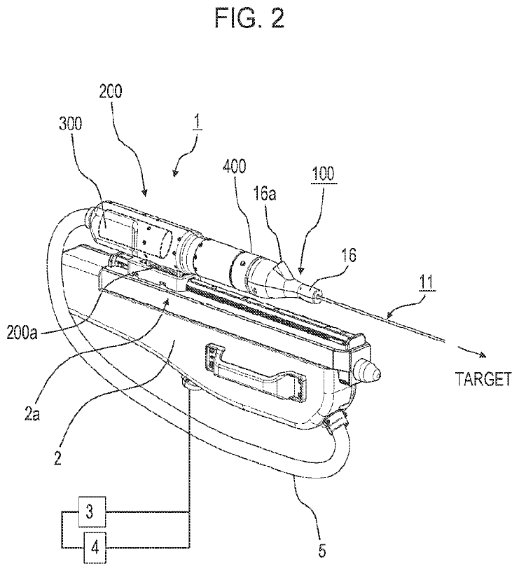

A medical system 1A and a medical device 1 will be described with reference to FIGS. 1 and 2. FIG. 1 is an overall view of the medical system 1A. FIG. 2 is a perspective view that shows the medical device 1 and a support 2.

The medical system 1A includes the medical device 1, the support 2 to which the medical device 1 is attached, and a controller 3 that controls the medical device 1. In the present embodiment, the medical system 1A includes a monitor 4 serving as a display apparatus.

The medical device 1 includes a catheter unit (bendable unit) 100 including a catheter 11 serving as a bendable body, and a base unit (a drive unit or an attached unit) 200. The catheter unit 100 is configured to be detachably attachable to the base unit 200.

In the present embodiment, a user of the medical system 1A and the medical device 1 is able to do some work, such as observing the inside of a target, collecting various samples from the inside of the target, and treating the inside of the target, by inserting the catheter 11 into the inside of the target. As one of embodiments, a user is able to insert the catheter 11 into the inside of a patient as a target. Specifically, by inserting the catheter 11 into a bronchus via the oral cavity or nasal cavity of a patient, it is possible to do some work, such as observation, collection, and removal of a lung tissue.

The catheter 11 can be used as a guide (sheath) that guides a medical tool for doing the above work. Examples of the medical tool (tool) include an endoscope, a forceps, and an ablation device. The catheter 11 itself may have the functions of the above-described medical tools.

In the present embodiment, the controller 3 includes a calculation device 3a and an input device 3b. The input device 3b receives a command or input for operating the catheter 11. The calculation device 3a includes a storage that stores a program and various data for controlling a catheter, a random access memory, and a central processing unit for running the program. The controller 3 may include an output unit that outputs a signal for displaying an image on the monitor 4.

As shown in FIG. 2, in the present embodiment, the medical device 1 is electrically connected to the controller 3 via the support 2 and a cable 5 that couples the base unit 200 of the medical device 1 with the support 2. The medical device 1 and the controller 3 may be directly connected by a cable. The medical device 1 and the controller 3 may be wirelessly connected to each other.

The medical device 1 is detachably attached to the support 2 via the base unit 200. More specifically, in the medical device 1, an attachment portion (connecting portion) 200a of the base unit 200 is detachably attached to a movable stage (receiving portion) 2a of the support 2. Even in a state where the attachment portion 200a of the medical device 1 is detached from the movable stage 2a, connection of the medical device 1 with the controller 3 is maintained such that the medical device 1 is controllable by the controller 3. In the present embodiment, even in a state where the attachment portion 200a of the medical device 1 is detached from the movable stage 2a, the medical device 1 and the support 2 are connected by the cable 5.

A user is able to manually move the medical device 1 in a state where the medical device 1 is detached from the support 2 (a state where the medical device 1 is detached from the movable stage 2a) and insert the catheter 11 into the inside of a target.

A user is able to use the medical device 1 in a state where the catheter 11 is inserted in a target and the medical device 1 is attached to the support 2. Specifically, when the movable stage 2a moves in a state where the medical device 1 is attached to the movable stage 2a, the medical device 1 moves. Then, an operation to move the catheter 11 in a direction to be inserted into the target and an operation to move the catheter 11 in a direction to be pulled out from the target are performed. Movement of the movable stage 2a is controlled by the controller 3.

The attachment portion 200a of the base unit 200 includes an unlock switch (not shown) and a detachment switch (not shown). In a state where the attachment portion 200a is attached to the movable stage 2a, a user is able to manually move the medical device 1 along a guide direction of the movable stage 2a while holding down the unlock switch. In other words, the movable stage 2a includes a guide configuration to guide movement of the medical device 1. When the user stops pressing the unlock switch, the medical device 1 is fixed to the movable stage 2a. On the other hand, when the detachment switch is pressed in a state where the attachment portion 200a is attached to the movable stage 2a, a user is able to detach the medical device 1 from the movable stage 2a.

A single switch may have the function of the unlock switch and the function of the detachment switch. When the unlock switch is provided with a mechanism of switching between a pressed down state and a non-pressed down state, a user does not need to hold down the unlock switch when manually sliding the medical device 1.

In a state where the attachment portion 200a is attached to the movable stage 2a and the unlock switch or the detachment switch is not pressed, the medical device 1 is fixed to the movable stage 2a and is moved by the movable stage 2a driven by a motor (not shown).

The medical device 1 includes a wire drive portion (a linear member drive portion, a line drive portion, or a main body drive portion) 300 for driving the catheter 11. In the present embodiment, the medical device 1 is a robot catheter device that drives the catheter 11 with the wire drive portion 300 controlled by the controller 3.

The controller 3 can control the wire drive portion 300 and perform an operation to bend the catheter 11. In the present embodiment, the wire drive portion 300 is incorporated in the base unit 200. More specifically, the base unit 200 includes a base housing 200f that accommodates the wire drive portion 300. In other words, the base unit 200 includes the wire drive portion 300. The wire drive portion 300 and the base unit 200 may be collectively referred to as a catheter drive apparatus (a base apparatus or a main body).

In an extending direction of the catheter 11, an end portion at which the distal end of the catheter 11 to be inserted into a target is disposed is referred to as a distal end. In the extending direction of the catheter 11, an opposite side to the distal end is referred to as a proximal end.

The catheter unit 100 has a proximal end cover 16 that covers the proximal end side of the catheter 11. The proximal end cover 16 has a tool hole 16a. A medical tool is allowed to be inserted into the catheter 11 via the tool hole 16a.

As described above, in the present embodiment, the catheter 11 has the function of a guide device for guiding a medical tool to a desired position inside a target.

For example, in a state where an endoscope is inserted in the catheter 11, the catheter 11 is inserted to an intended position inside a target. At this time, at least one of manual operation of a user, movement of the movable stage 2a, and drive of the catheter 11 with the wire drive portion 300 is used. After the catheter 11 reaches the intended position, the endoscope is pulled out from the catheter 11 via the tool hole 16a. Then, a medical tool is inserted through the tool hole 16a, and some work, such as collecting various samples from the inside of the target and treating the inside of the target, is performed.

As will be described later, the catheter unit 100 is detachably attached to the catheter drive apparatus (a base apparatus or a main body), more specifically, the base unit 200. After the medical device 1 is used, a user is able to detach the catheter unit 100 from the base unit 200, attach a new catheter unit 100 to the base unit 200, and use the medical device 1 again.

As shown in FIG. 2, the medical device 1 includes an operating portion 400. In the present embodiment, the operating portion 400 is provided in the catheter unit 100. The operating portion 400 is operated by a user when the catheter unit 100 is fixed to the base unit 200 or when the catheter unit 100 is detached from the base unit 200.

By connecting the endoscope inserted in the catheter 11 with the monitor 4, the monitor 4 can display an image taken by the endoscope on the monitor 4. By connecting the monitor 4 with the controller 3, the status of the medical device 1 and information related to control over the medical device 1 can be displayed on the monitor 4. For example, the position of the catheter 11 inside a target or information related to navigation for the catheter 11 inside a target can be displayed on the monitor 4. The monitor 4 and both the controller 3 and the endoscope may be connected by wire or may be connected by wireless. The monitor 4 and the controller 3 may be directly connected via the support 2.

### <Catheter>

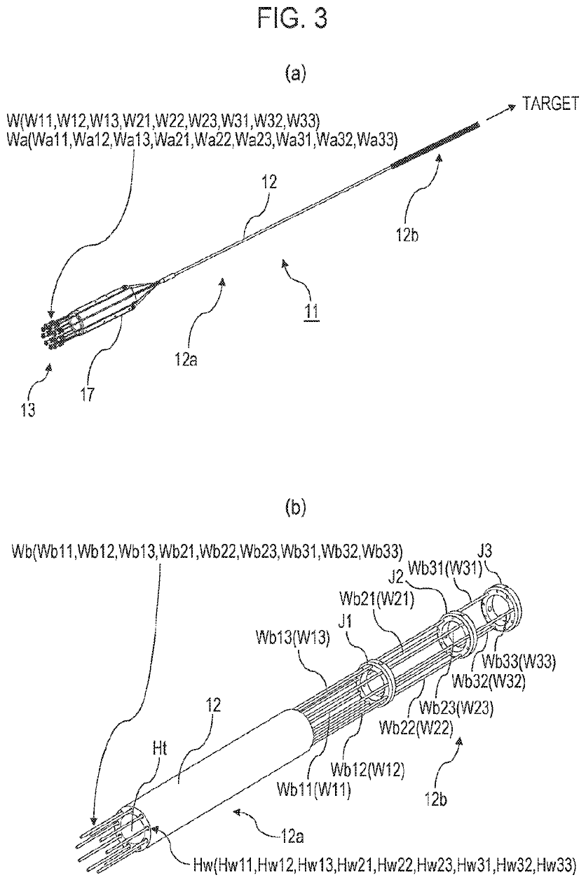

The catheter 11 serving as a bendable body will be described with reference to FIG. 3. FIG. 3 is a view that illustrates the catheter 11. FIG. 3(a) is a view that illustrates the whole of the catheter 11. FIG. 3(b) is an enlarged view of the catheter 11.

The catheter 11 includes a bendable portion (a bendable body or a catheter main body) 12 and a bend drive portion (catheter drive portion) 13 configured to bend the bendable portion 12. The bend drive portion 13 is configured to bend the bendable portion 12 upon receiving the driving force of the wire drive portion 300 via a coupling device 21 (described later).

The catheter 11 extends along the direction in which the catheter 11 is inserted into a target. The extending direction (longitudinal direction) of the catheter 11 is the same as an extending direction (longitudinal direction) of the bendable portion 12 and an extending direction (longitudinal direction) of each of first to ninth drive wires (W11 to W33) (described later).

The bend drive portion 13 includes the plurality of drive wires (drive lines, linear members, or linear actuators) connected to the bendable portion 12. Specifically, the bend drive portion 13 includes the first drive wire W11, the second drive wire W12, the third drive wire W13, the fourth drive wire W21, the fifth drive wire W22, the sixth drive wire W23, the seventh drive wire W31, the eighth drive wire W32, and the ninth drive wire W33.

Each of the first to ninth drive wires (W11 to W33) includes a held portion (a held shaft or a rod) Wa. Specifically, the first drive wire W11 includes the first held portion Wa11. The second drive wire W12 includes the second held portion Wa12. The third drive wire W13 includes the third held portion Wa13. The fourth drive wire W21 includes the fourth held portion Wa21. The fifth drive wire W22 includes the fifth held portion Wa22. The sixth drive wire W23 includes the sixth held portion Wa23. The seventh drive wire W31 includes the seventh held portion Wa31. The eighth drive wire W32 includes the eighth held portion Wa32. The ninth drive wire W33 includes the ninth held portion Wa33.

In the present embodiment, each of the first to ninth held portions (Wa11 to Wa33) has the same shape.

Each of the first to ninth drive wires (W11 to W33) includes a flexible wire body (a line body or a linear body) Wb. Specifically, the first drive wire W11 includes the first wire body Wb11. The second drive wire W12 includes the second wire body Wb12. The third drive wire W13 includes the third wire body Wb13. The fourth drive wire W21 includes the fourth wire body Wb21. The fifth drive wire W22 includes the fifth wire body Wb22. The sixth drive wire W23 includes the sixth wire body Wb23. The seventh drive wire W31 includes the seventh wire body Wb31. The eighth drive wire W32 includes the eighth wire body Wb32. The ninth drive wire W33 includes the ninth wire body Wb33.

In the present embodiment, each of the first to third wire bodies (Wb11 to Wb13) has the same shape and equal length. Each of the fourth to sixth wire bodies (Wb21 to Wb23) has the same shape and equal length. Each of the seventh to ninth wire bodies (Wb31 to Wb33) has the same shape and equal length. In the present embodiment, the first to third wire bodies (Wb11 to Wb13), the fourth to sixth wire bodies (Wb21 to Wb23), and the seventh to ninth wire bodies (Wb31 to Wb33) also have the same shape except for the length.

Each of the first to ninth held portions (Wa11 to Wa33) is fixed to a corresponding one of the first to ninth wire bodies (Wb11 to Wb33) at the proximal end of the corresponding one of the first to ninth wire bodies (Wb11 to Wb33).

The first to ninth drive wires (W11 to W33) are inserted into the bendable portion 12 via a wire guide 17 and fixed.

In the present embodiment, the material of each of the first to ninth drive wires (W11 to W33) is a metal. The material of each of the first to ninth drive wires (W11 to W33) may be a resin. The material of each of the first to ninth drive wires (W11 to W33) may include metal and resin.

Of the first to ninth drive wires (W11 to W33), a selected one may be referred to as a drive wire W. In the present embodiment, each of the first to ninth drive wires (W11 to W33) has the same shape except for the length of each of the first to ninth drive wires (Wb11 to Wb33).

In the present embodiment, the bendable portion 12 has flexibility and is a tubular member having a passage Ht for inserting a medical tool.

A wall of the bendable portion 12 includes a plurality of wire holes for respectively passing the first to ninth drive wires (W11 to W33). Specifically, the wall of the bendable portion 12 includes the first wire hole Hw11, the second wire hole Hw12, the third wire hole Hw13, the fourth wire hole Hw21, the fifth wire hole Hw22, the sixth wire hole Hw23, the seventh wire hole Hw31, the eighth wire hole Hw32, and the ninth wire hole Hw33. The first to ninth wire holes Hw (Hw11 to Hw33) are respectively in correspondence with the first to ninth drive wires (W11 to W33). The numeral suffixed to the sign Hw represents the numeral of a corresponding one of the drive wires. For example, the first drive wire W11 is inserted into the first wire hole Hw11.

Of the first to ninth wire holes (Hw11 to Hw33), a selected one may be referred to as a wire hole Hw. In the present embodiment, each of the first to ninth wire holes (Hw11 to Hw33) has the same shape.

The bendable portion 12 has an intermediate region 12a and a bendable region 12b. The bendable region 12b is disposed at the distal end of the bendable portion 12. A first guide ring J1, a second guide ring J2, and a third guide ring J3 are disposed in the bendable region 12b. The bendable region 12b means a region capable of controlling the degree and direction of bending of the bendable portion 12 by moving the first guide ring J1, the second guide ring J2, and the third guide ring J3 with the bend drive portion 13. FIG. 3(b) is a view drawn by omitting part of the bendable portion 12 covering the first to third guide rings (J1 to J3).

In the present embodiment, the bendable portion 12 includes a plurality of sub-rings (not shown). In the bendable region 12b, the first guide ring J1, the second guide ring J2, and the third guide ring J3 are fixed to the wall of the bendable portion 12. In the present embodiment, the sub-rings are respectively disposed between the first guide ring J1 and the second guide ring J2, and between the second guide ring J2 and the third guide ring J3.

A medical tool is guided to the distal end of the catheter 11 by the passage Ht, the first to third guide rings (J1 to J3), and the plurality of sub-rings.

Each of the first to ninth drive wires (W11 to W33) is fixed to a corresponding one of the first to third guide rings (J1 to J3) through the intermediate region 12a.

Specifically, the first drive wire W11, the second drive wire W12, and the third drive wire W13 are fixed to the first guide ring J1. The fourth drive wire W21, the fifth drive wire W22, and the sixth drive wire W23 extend through the first guide ring J1 and the plurality of sub-rings and are fixed to the second guide ring J2. The seventh drive wire W31, the eighth drive wire W32, and the ninth drive wire W33 extend through the first guide ring J1, the second guide ring J2, and the plurality of sub-rings and are fixed to the third guide ring J3.

The medical device 1 is capable of bending the bendable portion 12 in a direction that intersects with the extending direction of the catheter 11 by driving the bend drive portion 13 with the wire drive portion 300. Specifically, by moving each of the first to ninth drive wires (W11 to W33) in the extending direction of the bendable portion 12, the bendable region 12b of the bendable portion 12 is bent in a direction that intersects with the extending direction via the first to third guide rings (J1 to J3).

A user is able to insert the catheter 11 to an intended part inside a target by using at least any one of moving the medical device 1 manually or with the movable stage 2a and bending the bendable portion 12.

In the present embodiment, the bendable portion 12 is bent by moving the first to third guide rings (J1 to J3) with the first to ninth drive wires (W11 to W33); however, the present disclosure is not limited to this configuration. Any one or two of the first to third guide rings (J1 to J3) and the drive wires fixed to them may be omitted.

For example, the catheter 11 may have such a configuration that the seventh to ninth drive wires (W31 to W33) and the third guide ring J3 are provided and the first to sixth drive wires (W11 to W23) and the first and second guide rings (J1, J2) are omitted. Alternatively, the catheter 11 may have such a configuration that the fourth to ninth drive wires (W21 to W33) and the second and third guide rings (J2, J3) are provided and the first to third drive wires (W11 to W13) and the first guide ring J1 are omitted.

Alternatively, the catheter 11 may have such a configuration that a single guide ring is driven by two drive wires. In this case as well, the number of guide rings may be one or may be more than one.

### <Catheter Unit>

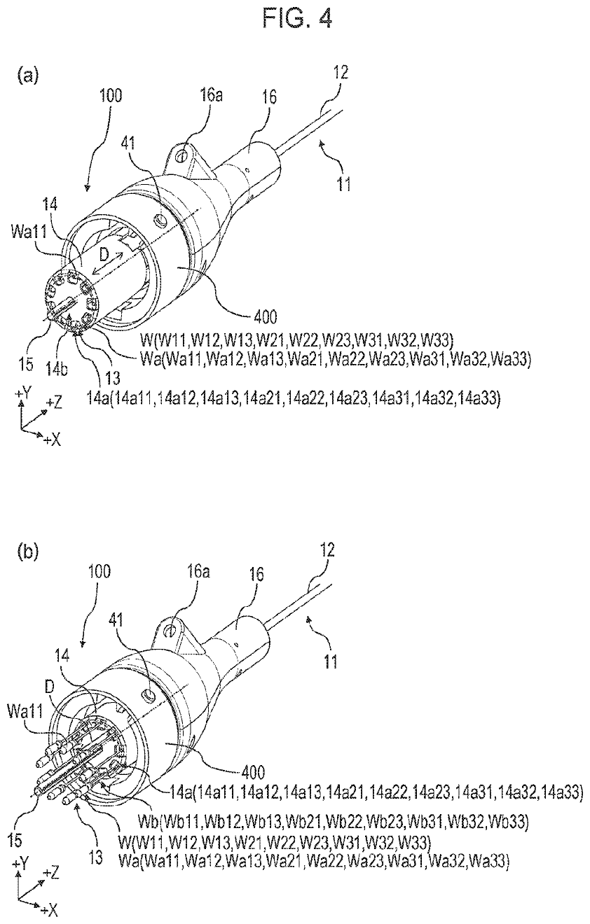

The catheter unit 100 will be described with reference to FIG. 4.

FIG. 4 is a view that illustrates the catheter unit 100. FIG. 4(a) is a view that illustrates the catheter unit 100 in a state where a wire cover 14 (described later) is at a cover position. FIG. 4(b) is a view that illustrates the catheter unit 100 in a state where the wire cover 14 (described later) is at an exposed position.

The catheter unit 100 includes the catheter 11 including the bendable portion 12 and the bend drive portion 13, and a proximal end cover 16 that supports the proximal end of the catheter 11. The catheter unit 100 includes the cover (wire cover) 14 for covering and protecting the first to ninth drive wires (W11 to W33) serving as the plurality of drive wires.

The catheter unit 100 is detachably attachable to the base unit 200 along an attaching and detaching direction DE. The direction in which the catheter unit 100 is attached to the base unit 200 and the direction in which the catheter unit 100 is detached from the base unit 200 are parallel to the attaching and detaching direction DE.

The proximal end cover (a frame, a bendable portion housing, or a catheter housing) 16 is a cover covering part of the catheter 11. The proximal end cover 16 has the tool hole 16a for inserting a medical tool into the passage Ht of the bendable portion 12.

The wire cover 14 has a plurality of exposure holes (wire cover holes or cover holes) for respectively passing the first to ninth drive wires (W11 to W33). The wire cover 14 has the first exposure hole 14a11, the second exposure hole 14a12, the third exposure hole 14a13, the fourth exposure hole 14a21, the fifth exposure hole 14a22, the sixth exposure hole 14a23, the seventh exposure hole 14a31, the eighth exposure hole 14a32, and the ninth exposure hole 14a33. The first to ninth exposure holes (14a11 to 14a33) are respectively in correspondence with the first to ninth drive wires (W11 to W33). The numeral suffixed to the sign 14a represents the numeral of a corresponding one of the drive wires. For example, the first drive wire W11 is inserted into the first exposure hole 14a11.

Of the first to ninth exposure holes (14a11 to 14a33), a selected one may be referred to as an exposure hole 14a. In the present embodiment, each of the first to ninth exposure holes (14a11 to 14a33) has the same shape.

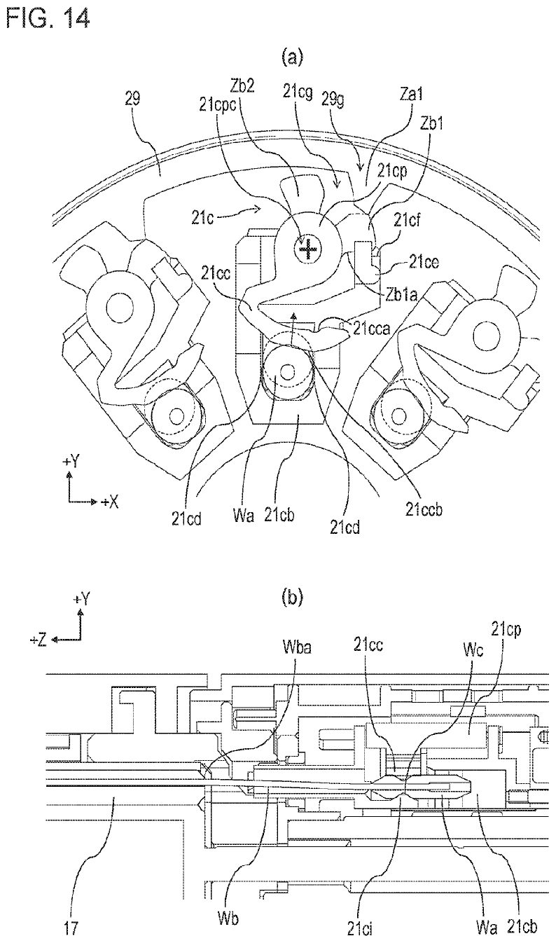

The wire cover 14 can be moved to a cover position (see FIG. 14(a)) where the first to ninth drive wires (W11 to W33) are covered and a cover retracted position (see FIG. 14(b)) retracted from the cover position. The cover retracted position may also be referred to as an exposed position where the first to ninth drive wires (W11 to W33) are exposed.

Before the catheter unit 100 is attached to the base unit 200, the wire cover 14 is located at the cover position. When the catheter unit 100 is attached to the base unit 200, the wire cover 14 moves from the cover position to the exposed position along the attaching and detaching direction DE.

In the present embodiment, after the wire cover 14 is moved from the cover position to the exposed position, the wire cover 14 is retained at the exposed position. Therefore, even when the catheter unit 100 is attached to the base unit 200 and then the catheter unit 100 is detached from the base unit 200, the wire cover 14 is retained at the exposed position.

However, after the wire cover 14 is moved from the cover position to the exposed position, the wire cover 14 may be configured to return to the cover position. For example, the catheter unit 100 may include an urging member that urges the wire cover 14 from the exposed position toward the cover position. In this case, when the catheter unit 100 is detached from the base unit 200 after the catheter unit 100 is attached to the base unit 200, the wire cover 14 is moved from the exposed position to the cover position.

When the wire cover 14 is at the exposed position, the first to ninth held portions (Wa11 to Wa33) of the first to ninth drive wires (W11 to W33) are exposed. As a result, coupling of the bend drive portion 13 with the coupling device 21 (described later) is allowed. When the wire cover 14 is at the exposed position, the first to ninth held portions (Wa11 to Wa33) of the first to ninth drive wires (W11 to W33) and parts of the wire bodies Wb protrude from the first to ninth exposure holes (14a11 to 14a33). More specifically, the first to ninth held portions (Wa11 to Wa33) protrude from the first to ninth exposure holes (14a11 to 14a33) in an attachment direction Da (described later).

As shown in FIG. 4(b), the first to ninth drive wires (W11 to W33) are arranged along a circle (imaginary circle) having a predetermined radius and are supported by the wire guide 17.

In the present embodiment, the catheter unit 100 includes a key shaft (a key or a catheter-side key) 15. In the present embodiment, the key shaft 15 extends in the attaching and detaching direction DE. The wire cover 14 has a shaft hole 14b through which the key shaft 15 extends. The key shaft 15 is engageable with a key receiving portion 22 (described later). When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

In the present embodiment, when viewed in the attaching and detaching direction DE, the first to ninth drive wires (W11 to W33) are disposed outside the key shaft 15 so as to surround the key shaft 15. In other words, the key shaft 15 is disposed inside the circle (imaginary circle) along which the first to ninth drive wires (W11 to W33) are arranged. Therefore, the key shaft 15 and the first to ninth drive wires (W11 to W33) can be disposed in a space-saving manner.

In the present embodiment, the catheter unit 100 includes the operating portion 400. The operating portion 400 is configured to be movable (rotatable) with respect to the proximal end cover 16 and the bend drive portion 13. The operating portion 400, the operating portion 400 is rotatable 400 around a rotation axis 400r. The rotation axis 400r of the operating portion 400 extends in the attaching and detaching direction DE.

In a state where the catheter unit 100 is attached to the base unit 200, the operating portion 400 is configured to be movable (rotatable) with respect to the base unit 200. More specifically, the operating portion 400 is configured to be movable (rotatable) with respect to the base housing 200f, the wire drive portion 300, and the coupling device 21 (described later).

In the present embodiment, as shown in FIG. 4, the extending direction (longitudinal direction) of the bendable portion 12 is defined as a positive Z direction. The direction of a straight line that is straight in the positive Z direction and that connects the center of the bendable portion 12 to the center of the engaged portion Wa11 is defined as a positive Y direction. The direction of a straight line that is straight in the positive Z direction and the positive Y direction is defined as a positive X direction.

### <Base Unit>

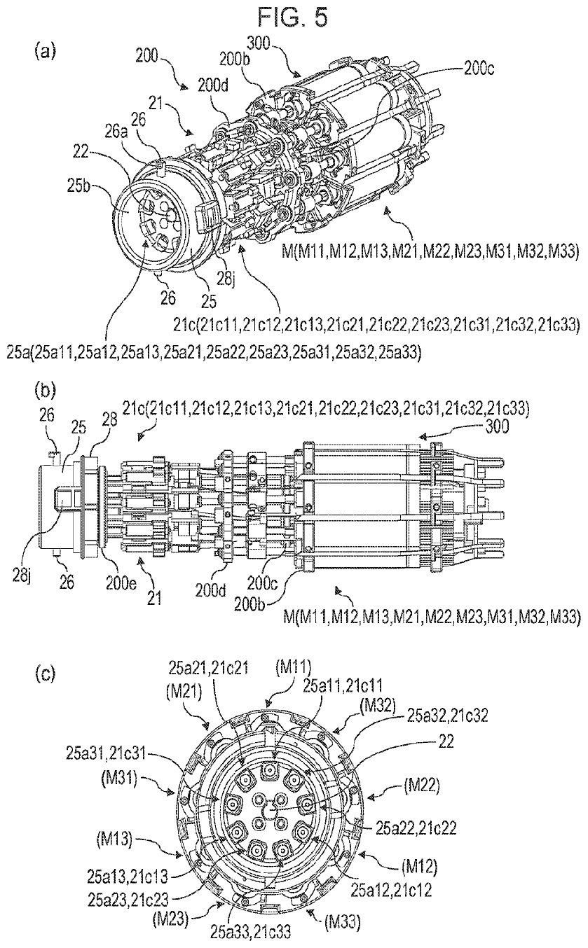

The base unit 200 and the wire drive portion 300 will be described with reference to FIG. 5.

FIG. 5 is a view that illustrates the base unit 200 and the wire drive portion 300. FIG. 5(a) is a perspective view that shows the internal structure of the base unit 200. FIG. 5(b) is a side view that shows the internal structure of the base unit 200. FIG. 5(c) is a view of the base unit 200 when viewed along the attaching and detaching direction DE.

As described above, the medical device 1 includes the base unit 200 and the wire drive portion 300. In the present embodiment, the wire drive portion 300 is accommodated in the base housing 200f and is provided inside the base unit 200. In other words, the base unit 200 includes the wire drive portion 300.

The wire drive portion 300 includes a plurality of driving sources (motors). In the present embodiment, the wire drive portion 300 includes the first driving source M11, the second driving source M12, the third driving source M13, the fourth driving source M21, the fifth driving source M22, the sixth driving source M23, the seventh driving source M31, the eighth driving source M32, and the ninth driving source M33.

Of the first to ninth driving sources (M11 to M33), a selected one may be referred to as a driving source M. In the present embodiment, each of the first to ninth driving sources (M11 to M33) has the same configuration.

The base unit 200 includes the coupling device 21. The coupling device 21 is accommodated in the base housing 200f. The coupling device 21 is connected to the wire drive portion 300. The coupling device 21 includes a plurality of coupling portions. In the present embodiment, the coupling device 21 includes the first coupling portion 21c11, the second coupling portion 21c12, the third coupling portion 21c13, the fourth coupling portion 21c21, the fifth coupling portion 21c22, the sixth coupling portion 21c23, the seventh coupling portion 21c31, the eighth coupling portion 21c32, and the ninth coupling portion 21c33.

Of the first to ninth coupling portions (21c11 to 21c33), a selected one may be referred to as a coupling portion 21c. In the present embodiment, each of the first to ninth coupling portions (21c11 to 21c33) has the same configuration.

Each of the plurality of coupling portions is connected to a corresponding one of the plurality of driving sources and is driven by the corresponding one of the plurality of driving sources. Specifically, the first coupling portion 21c11 is connected to the first driving source M11 and is driven by the first driving source M11. The second coupling portion 21c12 is connected to the second driving source M12 and is driven by the second driving source M12. The third coupling portion 21c13 is connected to the third driving source M13 and is driven by the third driving source M13. The fourth coupling portion 21c21 is connected to the fourth driving source M21 and is driven by the fourth driving source M21. The fifth coupling portion 21c22 is connected to the fifth driving source M22 and is driven by the fifth driving source M22. The sixth coupling portion 21c23 is connected to the sixth driving source M23 and is driven by the sixth driving source M23. The seventh coupling portion 21c31 is connected to the seventh driving source M31 and is driven by the seventh driving source M31. The eighth coupling portion 21c32 is connected to the eighth driving source M32 and is driven by the eighth driving source M32. The ninth coupling portion 21c33 is connected to the ninth driving source M33 and is driven by the ninth driving source M33.

As will be described later, the bend drive portion 13 including the first to ninth drive wires (W11 to W33) is coupled to the coupling device 21. The bend drive portion 13 receives the driving force of the wire drive portion 300 via the coupling device 21 to bend the bend drive portion 13.

The drive wire W is coupled to the coupling portion 21c via the held portion Wa. Each of the plurality of drive wires is coupled to a corresponding one of the plurality of coupling portions.

Specifically, the first held portion Wa11 of the first drive wire W11 is coupled to the first coupling portion 21c11. The second held portion Wa12 of the second drive wire W12 is coupled to the second coupling portion 21c12. The third held portion Wa13 of the third drive wire W13 is coupled to the third coupling portion 21c13. The fourth held portion Wa21 of the fourth drive wire W21 is coupled to the fourth coupling portion 21c21. The fifth held portion Wa22 of the fifth drive wire W22 is coupled to the fifth coupling portion 21c22. The sixth held portion Wa23 of the sixth drive wire W23 is coupled to the sixth coupling portion 21c23. The seventh held portion Wa31 of the seventh drive wire W31 is coupled to the seventh coupling portion 21c31. The eighth held portion Wa32 of the eighth drive wire W32 is coupled to the eighth coupling portion 21c32. The ninth held portion Wa33 of the ninth drive wire W33 is coupled to the ninth coupling portion 21c33.

The base unit 200 includes a base frame 25. The base frame 25 has a plurality of insertion holes for respectively passing the first to ninth drive wires (W11 to W33). The base frame 25 has the first insertion hole 25a11, the second insertion hole 25a12, the third insertion hole 25a13, the fourth insertion hole 25a21, the fifth insertion hole 25a22, the sixth insertion hole 25a23, the seventh insertion hole 25a31, the eighth insertion hole 25a32, and the ninth insertion hole 25a33. The first to ninth insertion holes (25a11 to 25a33) are respectively in correspondence with the first to ninth drive wires (W11 to W33). The numeral suffixed to the sign 25a represents the numeral of a corresponding one of the drive wires. For example, the first drive wire W11 is inserted into the first insertion hole 25a11.

Of the first to ninth insertion holes (25a11 to 25a33), a selected one may be referred to as an insertion hole 25a. In the present embodiment, each of the first to ninth insertion holes (25a11 to 25a33) has the same shape.

The base frame 25 has an attachment opening 25b into which the wire cover 14 is inserted. The first to ninth insertion holes (25a11 to 25a33) are disposed at the bottom portion of the attachment opening 25b.

In addition, the base unit 200 includes a motor frame 200b, a first bearing frame 200c, a second bearing frame 200d, and a third bearing frame 200e. The motor frame 200b, the first bearing frame 200c, the second bearing frame 200d, and the third bearing frame 200e are coupled to one another.

The base frame 25 has a key receiving portion (a key hole, a base-side key, or a main body-side key) 22 for receiving the key shaft 15. When the key shaft 15 and the key receiving portion 22 are engaged with each other, the catheter unit 100 is prevented from being attached to the base unit 200 in wrong phase.

When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

As a result, each of the first to ninth drive wires (W11 to W33) is engaged with a corresponding one of the first to ninth insertion holes (25a11 to 25a33) and a corresponding one of the first to ninth coupling portions (21c11 to 21c33). In other words, engagement of the drive wire W with the insertion hole 25a different from a corresponding one of the insertion holes 25a and coupling portion 21c different from a corresponding one of the coupling portions 21c is prevented.

A user is able to properly couple each of the first to ninth drive wires (W11 to W33) with a corresponding one of the first to ninth coupling portions (21c11 to 21c33) by engaging the key shaft 15 with the key receiving portion 22. Therefore, a user is able to easily attach the catheter unit 100 to the base unit 200.

In the present embodiment, the key shaft 15 has a protruding portion that protrudes in a direction to intersect with the attaching and detaching direction DE, and the key receiving portion 22 has a recess portion into which the protruding portion is inserted. A position in which the protruding portion and the recess portion are engaged with each other in the circumferential direction is a position in which each of the drive wires W is engaged with a corresponding one of the insertion holes 25a and a corresponding one of the coupling portions 21c.

The key shaft 15 may be disposed in any one of the base unit 200 and the catheter unit 100, and the key receiving portion 22 may be disposed in the other. For example, the key shaft 15 may be disposed at the base unit 200 side, and the key receiving portion 22 may be disposed at the catheter unit 100 side.

The base unit 200 has a joint 28 that includes a joint engagement portion 28j. The base frame 25 includes a lock shaft 26 having a lock protrusion 26a. The functions of them will be described later.

<Coupling of Motor with Drive Wire>

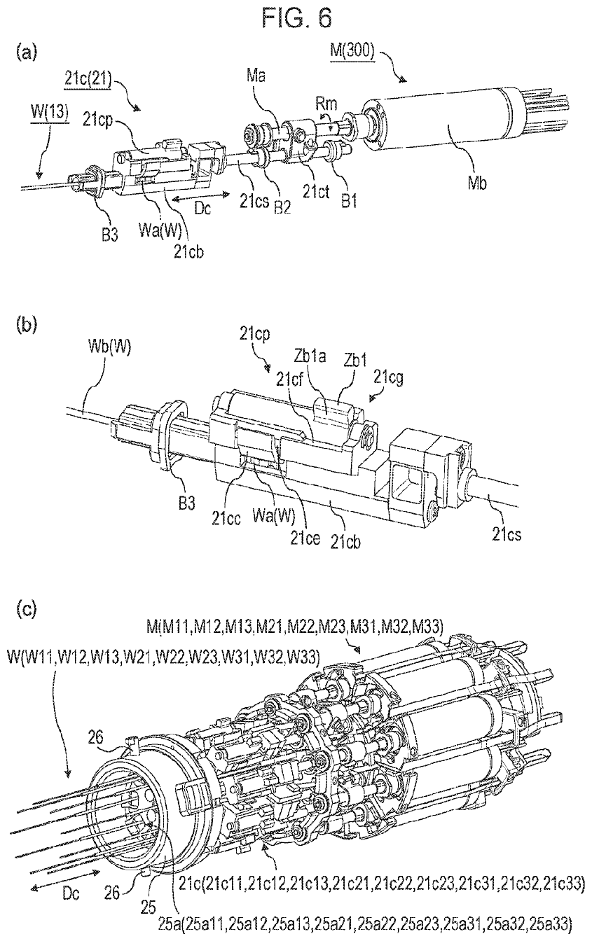

Coupling among the wire drive portion 300, the coupling device 21, and the bend drive portion 13 will be described with reference to FIG. 6.

FIG. 6 is a view that illustrates the wire drive portion 300, the coupling device 21, and the bend drive portion 13. FIG. 6(a) is a perspective view of the driving source M, the coupling portion 21c, and the drive wire W. FIG. 6(b) is an enlarged view of the coupling portion 21c and the drive wire W. FIG. 6(c) is a perspective view that shows coupling among the wire drive portion 300, the coupling device 21, and the bend drive portion 13.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth driving sources (M11 to M33) is the same. Therefore, in the following description, the configuration in which the drive wire W, the coupling portion 21c, and the driving source M are connected will be described by using the one drive wire W, the one coupling portion 21c, and the one driving source M.

As shown in FIGS. 6(a) and 6(b), the driving source M includes an output shaft Ma, and a motor main body Mb that rotates the output shaft Ma in a rotation direction Rm. A spiral groove is provided on the surface of the output shaft Ma. The output shaft Ma has a so-called screw shape. The motor main body Mb is fixed to the motor frame 200b.

The coupling portion 21c has a tractor 21ct connected to the output shaft Ma and a tractor support shaft 21cs that supports the tractor 21ct. The tractor support shaft 21cs is connected to a coupling base 21cb.

The coupling portion 21c includes a first rotary member 21cp for pressing the held portion Wa of the drive wire W. The drive wire W is engaged with the coupling portion 21c through the insertion hole 25a. As will be described in detail later, the first rotary member 21cp can be placed in a state where the held portion Wa is pressed against and engaged with the coupling base 21cb (fixed state) and a state where the held portion Wa is released (released state).

The first rotary member 21cp has a gear portion 21cg meshed with an internal gear 29 (described later) and a cam 21cc serving as a pressing portion for pressing the held portion Wa of the drive wire W.

As will be described later, the cam 21cc can move with respect to the held portion Wa. When the cam 21cc moves, the held portion Wa is switched between the fixed state and the released state.

The coupling portion 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. Therefore, when the motor shaft Ma rotates in the rotation direction Rm, rotation of the coupling portion 21c around the motor shaft Ma is restricted. The first bearing B1, the second bearing B2, and the third bearing B3 are provided for each of the first to ninth coupling portions (21c11 to 21c33).

Since rotation of the coupling portion 21c around the motor shaft Ma is restricted, when the motor shaft Ma rotates, a force along the rotational axis direction of the motor shaft Ma is applied to the tractor 21ct by the spiral groove of the motor shaft Ma. As a result, the coupling portion 21c moves along the rotational axis direction of the motor shaft Ma (Dc direction). When the coupling portion 21c moves, the drive wire W moves, and the bendable portion 12 bends.

In other words, the motor shaft Ma and the tractor 21ct make up a so-called feed screw that converts rotational motion transmitted from the driving source M to linear motion by a screw. In the present embodiment, the motor shaft Ma and the tractor 21ct are slide screws. Alternatively, the motor shaft Ma and the tractor 21ct may be ball screws.

As shown in FIG. 6(c), by attaching the catheter unit 100 to the base unit 200, each of the first to ninth drive wires (W11 to W33) and a corresponding one of the first to ninth coupling portions (21c11 to 21c33) are coupled to each other.

The controller 3 is capable of controlling the first to ninth driving sources (M11 to M33) independently of one another. In other words, a selected one driving source of the first to ninth driving sources (M11 to M33) is allowed to independently operate or stop regardless of whether the other driving sources are stopped. In other words, the controller 3 is capable of controlling each of the first to ninth drive wires (W11 to W33) independently of one another. As a result, each of the first to third guide rings (J1 to J3) is controlled independently of one another, and the bendable region 12b of the bendable portion 12 is allowed to bend in a selected direction.

### <Attachment of Catheter Unit>

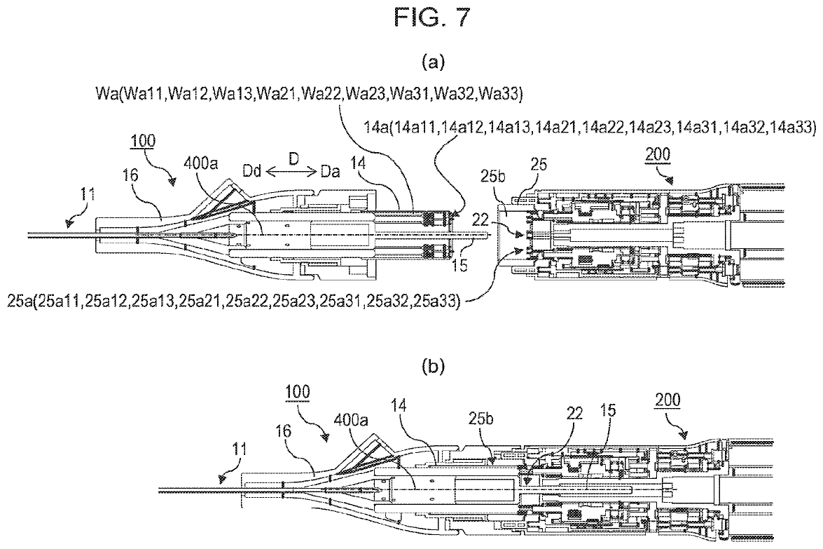

An operation to attach the catheter unit 100 to the base unit 200 will be described with reference to FIG. 7.

FIG. 7 is a view that illustrates attachment of the catheter unit 100. FIG. 7(a) is a view before the catheter unit 100 is attached to the base unit 200. FIG. 7(b) is a view after the catheter unit 100 is attached to the base unit 200.

In the present embodiment, the attaching and detaching direction DE of the catheter unit 100 is the same as the direction of the rotation axis 400r of the operating portion 400. In the attaching and detaching direction DE, the direction in which the catheter unit 100 is attached to the base unit 200 is referred to as the attachment direction Da. In the attaching and detaching direction DE, the direction in which the catheter unit 100 is detached from the base unit 200 (a direction opposite to the attachment direction Da) is referred to as a detachment direction Dd.

As shown in FIG. 7(a), in a state before the catheter unit 100 is attached to the base unit 200, the wire cover 14 is placed at the cover position. At this time, the wire cover 14 covers the first to ninth drive wires (W11 to W33) such that the first to ninth held portions (Wa11 to Wa33) do not protrude from the first to ninth exposure holes (14a11 to 14a33) of the wire cover 14. Therefore, in a state before the catheter unit 100 is attached to the base unit 200, the first to ninth drive wires (W11 to W33) are protected.

When the catheter unit 100 is attached to the base unit 200, the key shaft 15 is engaged with the key receiving portion 22. The key shaft 15 protrudes from the wire cover 14. In the present embodiment, in a state where the key shaft 15 has reached the entrance of the key receiving portion 22, the wire cover 14 is not engaged with the attachment opening 25b. In other words, when the phase of the catheter unit 100 with respect to the base unit 200 is a phase in which the key shaft 15 and the key receiving portion 22 are not engaged with each other, the wire cover 14 is not engaged with the attachment opening 25b, and the state where the wire cover 14 is placed at the cover position is maintained. Therefore, even when the catheter unit 100 is moved such that the key shaft 15 and the key receiving portion 22 are engaged with each other, the first to ninth drive wires (W11 to W33) are protected.

When the key shaft 15 and the key receiving portion 22 are engaged with each other and the catheter unit 100 is moved in the attachment direction Da with respect to the base unit 200, the catheter unit 100 is attached to the base unit 200. When the catheter unit 100 is attached to the base unit 200, the wire cover 14 is moved to the exposed position. In the present embodiment, the wire cover 14 contacts with the base frame 25 to move from the cover position to the exposed position (see FIG. 7(b)).

More specifically, when the catheter unit 100 is attached, the wire cover 14 contacts with the base frame 25 to stop. In this state, by moving the catheter unit 100 in the attachment direction Da, the wire cover 14 relatively moves with respect to a part other than the wire cover 14 in the catheter unit 100. As a result, the wire cover 14 moves from the cover position to the exposed position.

The wire cover 14 moves from the cover position to the exposed position, while the held portion Wa of the drive wire W protrudes from the exposure hole 14a of the wire cover 14 and is inserted into the insertion hole 25a. Then, the held portion Wa is engaged with the coupling base 21cb of the coupling portion 21c (see FIG. 6(b)).

In a state where the catheter unit 100 is just attached to the base unit 200, the catheter unit 100 can be detached by moving the catheter unit 100 in the detachment direction Dd with respect to the base unit 200. As will be described later, in a state where the catheter unit 100 is just attached to the base unit 200, fixing of the drive wire W with the coupling portion 21c is in an unlocked state.

In a state where the catheter unit 100 is attached to the base unit 200, detachment of the catheter unit 100 from the base unit 200 by operating the operating portion 400 is prevented. In addition, by operating the operating portion 400 in a state where the catheter unit 100 is attached to the base unit 200, the bend drive portion 13 is fixed to the coupling device 21, and the bend drive portion 13 is coupled to the wire drive portion 300 via the coupling device 21.

### <Fixing of Bend Drive Portion and Unlocking of Fixing>

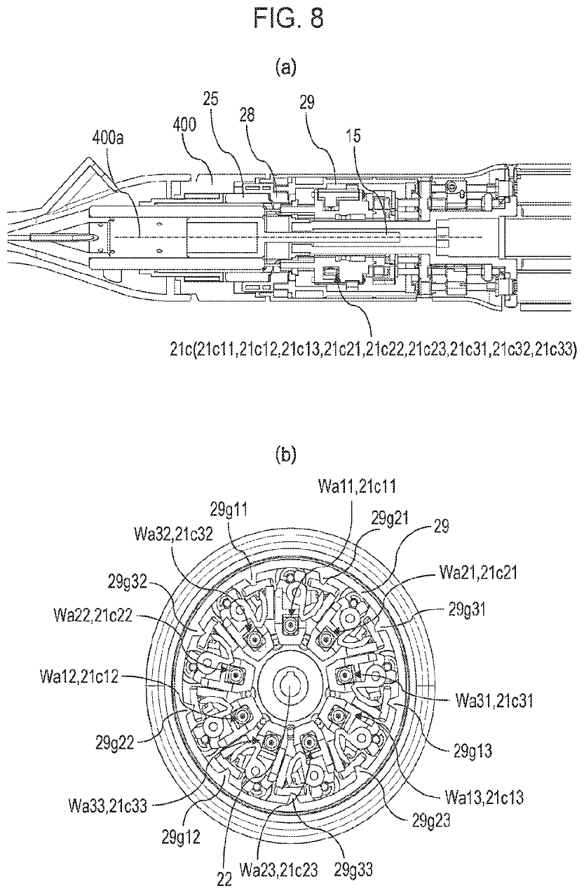

A configuration for fixing the bend drive portion 13 to the coupling device 21 and a configuration for unlocking fixing of the bend drive portion 13 with the coupling device 21 will be described with reference to FIGS. 8, 9, 10, 11, 12, 13, and 14.

FIG. 8 is a view that illustrates coupling of the catheter unit 100 with the base unit 200. FIG. 8(a) is a sectional view of the catheter unit 100 and the base unit 200. FIG. 8(a) is a sectional view of the catheter unit 100 and the base unit 200, taken along the rotation axis 400r. FIG. 8(b) is a sectional view of the base unit 200. FIG. 8(b) is a sectional view of the base unit 200, taken in a direction orthogonal to the rotation axis 400r at a part of the coupling portion 21c.

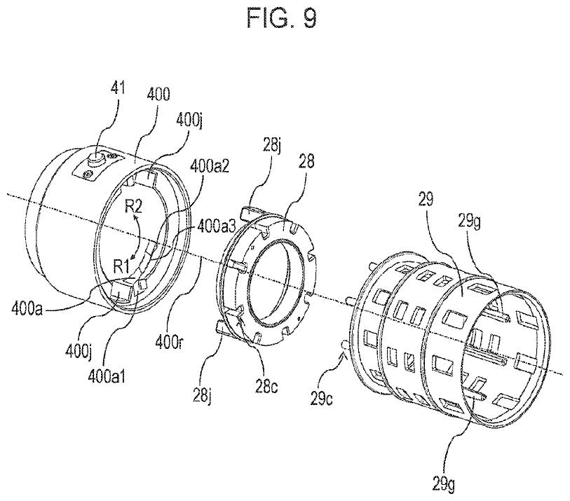

FIG. 9 is an exploded view that illustrates coupling of the catheter unit 100 with the base unit 200.

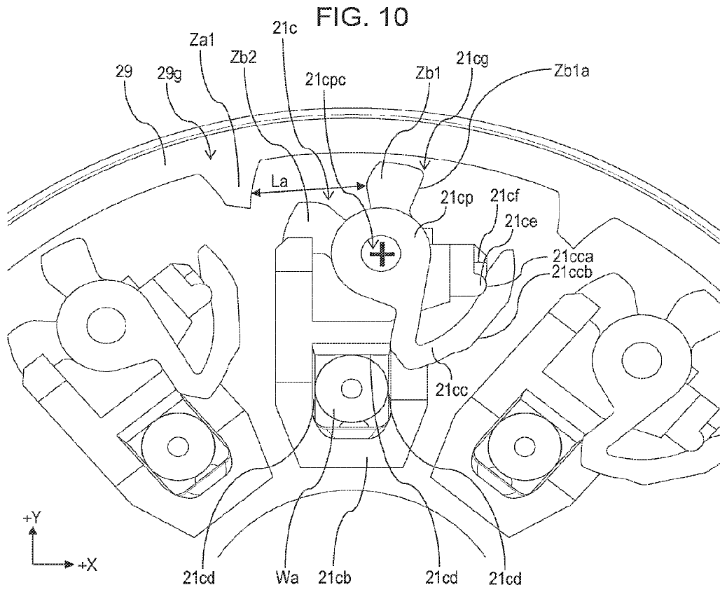

FIGS. 10, 11, 12, 13, and 14 are views that illustrate fixing of the drive wire W with the coupling portion 21c.

As shown in FIGS. 8(a) and 9, the base unit 200 includes the joint (an intermediate member or a second transmission member) 28, and the internal gear 29 serving as a movable gear (an interlocking gear, a transmission member, or a first transmission member) that is in interlocking with the operating portion 400 via the joint 28.

The joint 28 has a plurality of transmitting portions 28c. The internal gear 29 has a plurality of transmitted portions 29c. The plurality of transmitting portions 28c is engaged with the plurality of transmitted portions 29c, and, when the joint 28 rotates, rotation of the joint 28 is transmitted to the internal gear 29.

When the catheter unit 100 is attached to the base unit 200, the engagement portion 400j provided in the operating portion 400 is engaged with the joint engagement portion 28j of the joint 28. When the operating portion 400 rotates, rotation of the operating portion 400 is transmitted to the joint 28. The operating portion 400, the joint 28, and the internal gear 29 rotate in the same direction.

The internal gear 29 has a plurality of tooth portions for switching between a state where each of the first to ninth coupling portions (21cl1 to 21c33) fixes a corresponding one of the first to ninth drive wires (W11 to W33) and a state where each of the first to ninth coupling portions (21c11 to 21c33) releases a corresponding one of the first to ninth drive wires (W11 to W33). Each of the plurality of tooth portions (a working portion or a gear switching portion) of the internal gear 29 is engaged with the gear portion 21cg of the first rotary member 21cp of a corresponding one of the first to ninth coupling portions (21c11 to 21c33).

Specifically, in the present embodiment, the internal gear 29 has the first tooth portion 29g11, the second tooth portion 29g12, the third tooth portion 29g13, the fourth tooth portion 29g21, the fifth tooth portion 29g22, the sixth tooth portion 29g23, the seventh tooth portion 29g31, the eighth tooth portion 29g32, and the ninth tooth portion 29g33. The first to ninth tooth portions (29g11 to 29g33) are formed with a gap from each other.

The first tooth portion 29g11 meshes with the gear portion 21cg of the first coupling portion 21c11. The second tooth portion 29g12 meshes with the gear portion 21cg of the second coupling portion 21c12. The third tooth portion 29g13 meshes with the gear portion 21cg of the third coupling portion 21c13. The fourth tooth portion 29g21 meshes with the gear portion 21cg of the fourth coupling portion 21c21. The fifth tooth portion 29g22 meshes with the gear portion 21cg of the fifth coupling portion 21c22. The sixth tooth portion 29g23 meshes with the gear portion 21cg of the sixth coupling portion 21c23. The seventh tooth portion 29g31 meshes with the gear portion 21cg of the seventh coupling portion 21c31. The eighth tooth portion 29g32 meshes with the gear portion 21cg of the eighth coupling portion 21c32. The ninth tooth portion 29g33 meshes with the gear portion 21cg of the ninth coupling portion 21c33.

Of the first to ninth tooth portions (29g11 to 29g33), a selected one may be referred to as a tooth portion 29g. In the present embodiment, each of the first to ninth tooth portions (29g11 to 29g33) has the same configuration. In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth tooth portions (29g11 to 29g33) is the same. Therefore, in the following description, the configuration in which the drive wire W, the coupling portion 21c, and the tooth portion 29g are connected will be described by using the one drive wire W, the one coupling portion 21c, and the one tooth portion 29g.

In each of the first to ninth coupling portions (21c11 to 21c33), when the gear portion 21cg is moved by the internal gear 29, the first rotary member 21cp rotates, and the cam 21cc moves to a pressing position and a retracted position retracted from the pressing position. The state where the cam 21cc is moved to the pressing position is shown in FIG. 14(b), and the state where the cam 21cc is moved to the retracted position is shown in FIG. 10.

By rotating the operating portion 400, the internal gear 29 rotates. When the internal gear 29 rotates, each of the first to ninth coupling portions (21c11 to 21c33) operates. In other words, with an operation to rotate the one operating portion 400, the first to ninth coupling portions (21c11 to 21c33) are actuated.

The operating portion 400 is allowed to move to a fixed position (lock position) and a detachment position in a state where the catheter unit 100 is attached to the base unit 200. As will be described later, the operating portion 400 is allowed to move to the unlock position in a state where the catheter unit 100 is attached to the base unit 200. In the circumferential direction of the operating portion 400, the unlock position is located between the fixed position and the detachment position. In a state where the operating portion 400 is placed at the detachment position, the catheter unit 100 is attached to the base unit 200.

In a state where the catheter unit 100 is attached to the base unit 200, fixing (locking) of the drive wire W to the coupling portion 21c is unlocked. This state is referred to as an unlocked state of the coupling portion 21c. A state where the drive wire W is fixed (locked) to the coupling portion 21c is referred to as a locked state of the coupling portion 21c.

An operation at the time of fixing the drive wire W to the coupling portion 21c will be described with reference to FIGS. 10, 11, 12, 13, and 14.

In a state after the catheter unit 100 is attached to the base unit 200 and before the operating portion 400 is operated, the catheter unit 100 is allowed to be detached from the base unit 200. Hereinafter, a state where the catheter unit 100 is allowed to be detached from the base unit 200 is referred to as a detachable state.

FIG. 10 is a view that shows a state of the internal gear 29 and the coupling portion 21c in the detachable state. FIG. 10 is a view that shows the internal gear 29 and the coupling portion 21c in a state where the operating portion 400 is placed at the fixed position.

The coupling base 21cb includes a cam portion holding portion 21ce and a plurality of rod support surfaces 21cd that each support the held portion Wa of the drive wire W. The cam 21cc provided on the first rotary member 21cp has a holding surface 21cca and a pressing surface 21ccb.

As shown in FIG. 10, in the detachable state (a state where the operating portion 400 is at the detachment position), the first rotary member 21cp is held at a position at which the holding surface 21cca is engaged with the cam portion holding portion 21ce of the coupling base 21cb. A tooth Za1 of the internal gear 29 and a tooth Zb1 of the gear portion 21cg are stopped in a state where there is a clearance La therebetween.

In the rotation direction of the operating portion 400, a direction in which the operating portion 400 heads from the detachment position for the unlock position and the fixed position is referred to as a lock direction (fixing direction), and a direction in which the operating portion 400 heads from the fixed position for the unlock position and the detachment position is referred to as an unlock direction. The operating portion 400 rotates in the unlock direction from the unlock position and moves to the detachment position. The operating portion 400 rotates in the lock direction from the unlock position to move to the fixed position.

In a state where the catheter unit 100 is attached to the base unit 200 and the operating portion 400 is at the detachment position, the coupling portion 21c is in an unlocked state, and fixing of the drive wire W with the coupling portion 21c is unlocked. At this time, the held portion Wa is supported by the rod support surface 21cd provided on the coupling base 21cb, and no rod support surface 21cd is present only in a negative Y direction. Thus, the held portion Wa is supported so as to be movable only in the negative Y direction.

When the coupling portion 21c is in the unlocked state, the cam 21cc is located at a position with a clearance from the held portion Wa. In other words, fixing of the held portion Wa is unlocked.

When the catheter unit 100 is moved in the detachment direction Dd with respect to the base unit 200 while the coupling portion 21c is in the unlocked state, the held portion Wa can be pulled out from the coupling base 21cb.

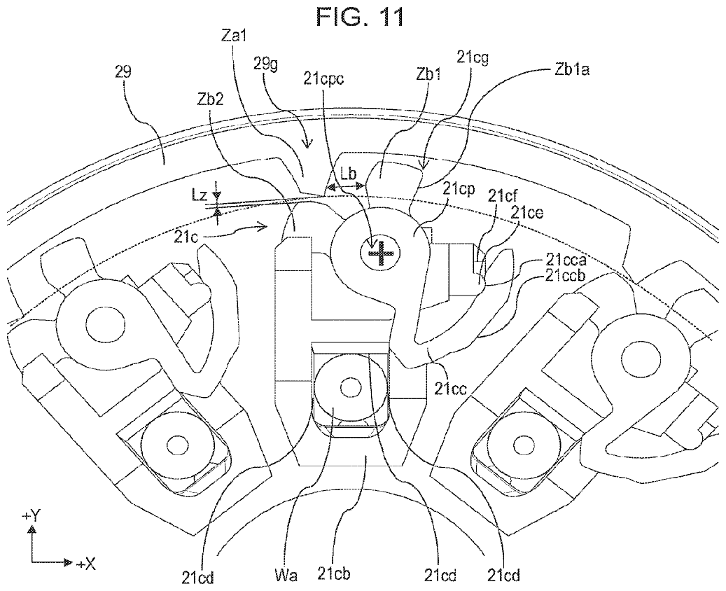

FIG. 11 is a view that shows a state of the internal gear 29 and the coupling portion 21c when the operating portion 400 is rotated in the lock direction from the detachment position. FIG. 11 is a view that shows a state of the internal gear 29 and the coupling portion 21c in a state where the operating portion 400 is at the unlock position.

When the operating portion 400 is rotated in the lock direction in a state where the operating portion 400 is at the detachment position (FIG. 10), the internal gear 29 rotates in the clockwise direction. Then, the operating portion 400 is placed at the unlock position.

Even when the operating portion 400 is rotated, the key shaft 15 and the key receiving portion 22 are engaged with each other, so rotation of the whole (except the operating portion 400) of the catheter unit 100 with respect to the base unit 200 is restricted. In other words, the operating portion 400 is rotatable with respect to the whole (except the operating portion 400) of the catheter unit 100 and the base unit 200 in a state where the whole (except the operating portion 400) of the catheter unit 100 and the base unit 200 are stopped.

When the internal gear 29 rotates in the clockwise direction, the clearance between the tooth Za1 of the internal gear 29 and the tooth Zb1 of the gear portion 21cg reduces from a clearance La to a clearance Lb.

A tooth Zb2 of the gear portion 21cg is disposed at a position spaced a clearance Lz apart from a tip circle (dashed line) of the tooth portion 29g of the internal gear 29. Therefore, the internal gear 29 is rotatable without interference with the tooth Zb2. On the other hand, the coupling portion 21c is maintained in the same state (unlocked state) as the state shown in FIG. 10.

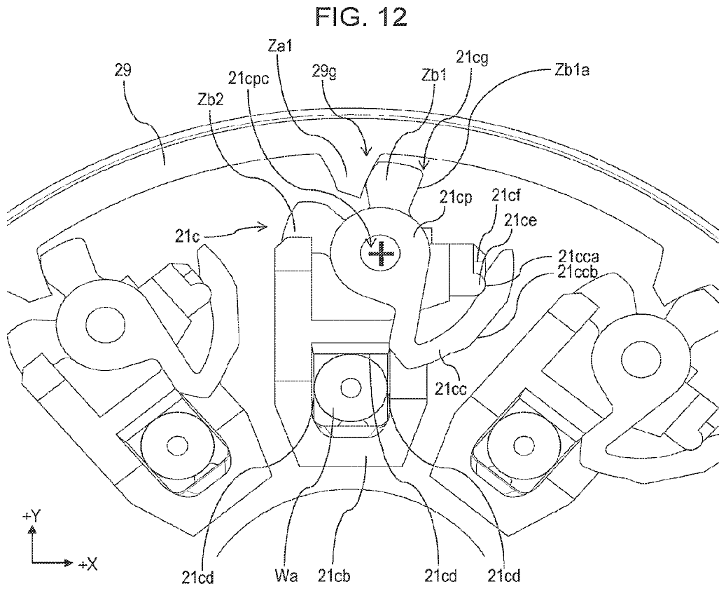

When the operating portion 400 is further rotated from the state shown in FIG. 11 in the lock direction, the internal gear 29 is further rotated in the clockwise direction. FIG. 12 shows a state of the internal gear 29 and the coupling portion 21c at that time.

FIG. 12 is a view that shows a state of the internal gear 29 and the coupling portion 21c when the operating portion 400 is rotated in the lock direction from the unlock position.

As shown in FIG. 12, when the operating portion 400 is rotated in the lock direction from the unlock position, the tooth Za1 of the internal gear 29 and the tooth Zb1 of the gear portion 21cg contact with each other. On the other hand, the coupling portion 21c is maintained in the unlocked state that is the same state as the state shown in FIGS. 10 and 11.

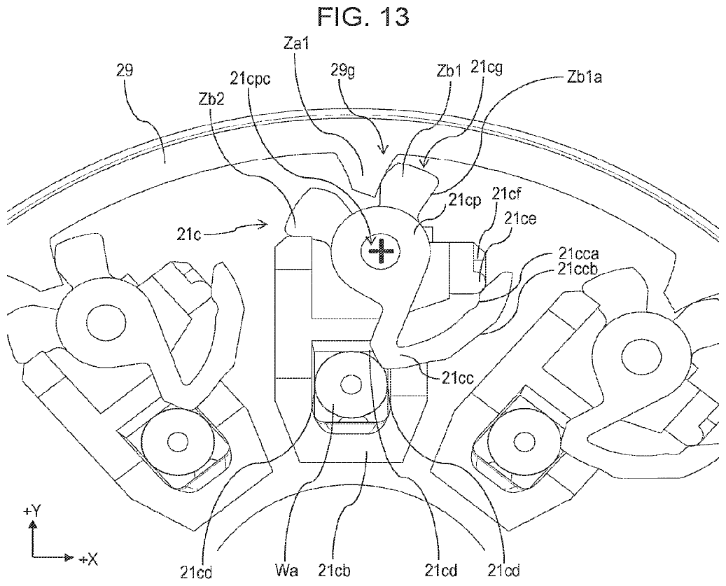

When the operating portion 400 is further rotated from the state shown in FIG. 12 in the lock direction, the internal gear 29 is further rotated in the clockwise direction. FIG. 13 shows a state of the internal gear 29 and the coupling portion 21c at that time.

FIG. 13 is a view that shows a state of the internal gear 29 and the coupling portion 21c when the operating portion 400 is rotated in the lock direction from the state shown in FIG. 12.

As shown in FIG. 13, when the operating portion 400 is further rotated from the state shown in FIG. 12 in the lock direction, the internal gear 29 is further rotated in the clockwise direction.

The internal gear 29 shifts from the state of FIG. 12 to the state of FIG. 13, with the result that the internal gear 29 rotates the gear portion 21cg in the clockwise direction. When the gear portion 21cg rotates, engagement of the holding surface 21cca with the cam portion holding portion 21ce is unlocked, and the cam 21cc contacts with the held portion Wa. Therefore, the held portion Wa begins to receive a force from the cam 21cc.

FIG. 14(a) is a view that shows a state of the internal gear 29 and the coupling portion 21c when the operating portion 400 is rotated in the lock direction from the state shown in FIG. 13.

As shown in FIG. 14(a), when the operating portion 400 is further rotated from the state shown in FIG. 13 in the lock direction, the internal gear 29 is further rotated in the clockwise direction. The internal gear 29 shifts from the state of FIG. 13 to the state of FIG. 14(a), with the result that the internal gear 29 rotates the gear portion 21cg in the clockwise direction. A rotational trajectory of the pressing surface 21ccb at the time when the gear portion 21cg rotates overlaps the position (dashed line) of the held portion Wa in the state shown in FIG. 13. Thus, when the gear portion 21cg rotates, the pressing surface 21ccb rotates while interfering with the held portion Wa. At this time, since the held portion Wa is supported so as to be movable only in the negative Y direction by a rod support surfaces provided in the coupling base 21cb, the held portion Wa is pushed by the pressing surface 21ccb to move in the negative Y direction.

FIG. 14(b) is a sectional view that shows a state of the internal gear 29 and the coupling portion 21c shown in FIG. 14(a). As shown in FIG. 14(b), when the held portion Wa moves in the negative Y direction, the wire body Wb that is a flexible member supported by the wire guide 17 elastically deforms. Specifically, the held portion Wa placed in a cantilever state deflects with a wire body support end Wba supported by the wire guide 17 as a fulcrum. When the held portion Wa moves in the negative Y direction, a held portion recess Wc provided in the held portion Wa and a coupling base protrusion 21ci provided in the coupling base 21cb are engaged with each other. Thus, the held portion Wa is fixed to the coupling base 21cb.

The rotational trajectory of the pressing surface 21ccb also overlaps the position of the held portion Wa fixed, and the cam 21cc elastically deforms by the amount of interference. Thus, the held portion Wa is firmly fixed to the coupling base 21cb by the elastic force of the cam 21cc. When the tooth Za1 of the internal gear 29 separates from the tooth Zb1 of the gear portion 21cg, transmission of driving force from the internal gear 29 to the gear portion 21cg stops. At this time, since the cam 21cc is elastically deformed, the cam 21cc receives a reaction force from the held portion Wa fixed. In the radial direction of rotation of the first rotary member 21cp, the reaction force applied to the cam 21cc is applied at a position spaced apart from the rotation center 21cpc of the first rotary member 21cp, so the first rotary member 21cp rotates in the clockwise direction. At this time, the first rotary member 21cp rotates in the same direction as the direction to be rotated by the internal gear 29 that rotates in the clockwise direction.

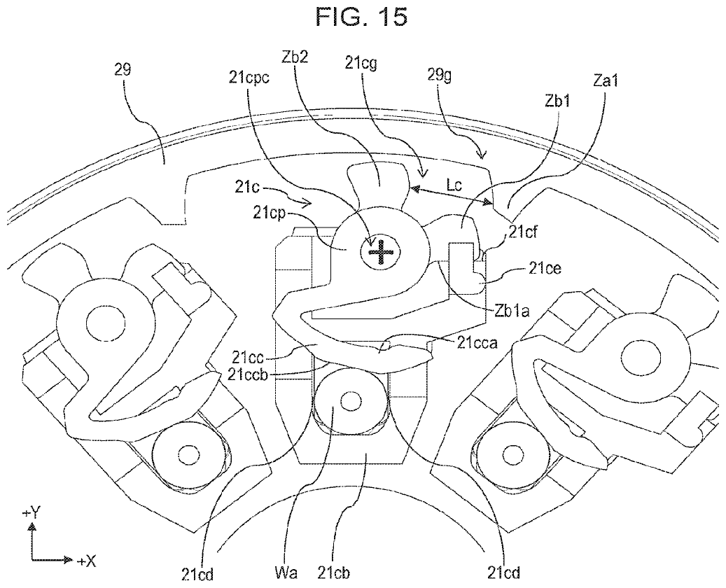

FIG. 15 is a view that shows a state of the internal gear 29 and the coupling portion 21c when the operating portion 400 is rotated in the lock direction from the state shown in FIG. 14(a).

As shown in FIG. 15, from the state shown in FIG. 14(a), the cam 21cc provided on the first rotary member 21cp receives a reaction force, and the first rotary member 21cp further rotates.

As shown in FIG. 15, after the first rotary member 21cp rotates, the first rotary member 21cp rotates until a stop surface Zb1a provided on the tooth Zb1 contacts with a stopped surface 21cf provided on the coupling base 21cb. In other words, the first rotary member 21cp stops in a state where the stop surface Zb1a and the stopped surface 21cf are arranged in the same plane.

At this time, the coupling portion 21c is in a locked state. When the coupling portion 21c is in the locked state, the cam 21cc of the first rotary member 21cp is placed at the pressing position, and the pressing surface 21ccb presses the held portion Wa against the coupling base 21cb. In the pressed state, as shown in FIG. 14(b), the held portion recess Wc and the coupling base protrusion 21ci are engaged with each other, so the held portion Wa is stably fixed. When the coupling portion 21c is in the locked state, the held portion Wa is restricted from being pulled out from the coupling base 21cb.

The tooth Za1 of the internal gear 29 and the tooth Zb2 of the gear portion 21cg are stopped at positions where there is a clearance Lc therebetween.

When fixing of the drive wire W to the coupling portion 21c is unlocked, the operating portion 400 placed at the fixed position is rotated in the unlock direction. At this time, the internal gear 29 rotates from the state shown in FIG. 15 in the counterclockwise direction. When the internal gear 29 rotates in the counterclockwise direction, the tooth Za1 of the internal gear 29 contacts with the tooth Zb2 of the gear portion 21cg, and the first rotary member 21cp is rotated in the counterclockwise direction.

By further rotating the internal gear 29 in the counterclockwise direction, fixing of the drive wire W with the coupling portion 21c is unlocked. The operations of the internal gear 29 and the first rotary member 21cp at this time are operations reverse to the above-described operations. In other words, fixing of the drive wire W with the coupling portion 21c is unlocked by the operation reverse to the operation at the time of fixing the above-described drive wire W with the coupling portion 21c.

The above-described operations are performed in each of the first to ninth coupling portions (21c11 to 21c33). In other words, in the course in which the operating portion 400 moves from the detachment position to the fixed position, the first to ninth coupling portions (21c11 to 21c33) shift from the unlocked state to the locked state by movement (rotation) of the operating portion 400. In the course in which the operating portion 400 moves from the fixed position to the detachment position, the first to ninth coupling portions (21c11 to 21c33) shift from the locked state to the unlocked state by movement (rotation) of the operating portion 400. In other words, a user is able to switch the plurality of coupling portions between the unlocked state and the locked state by operating the single operating portion 400.

In other words, it is not necessary that each of the plurality of coupling portions includes an operating portion for switching between the unlocked state and the locked state and a user operates the plurality of coupling portions. Therefore, a user is able to easily attach and detach the catheter unit 100 to the base unit 200. In addition, the medical device 1 is simplified.

A state where each of the first to ninth drive wires (W11 to W33) is fixed by a corresponding one of the first to ninth coupling portions (21c11 to 21c33) is referred to as a first state. A state where fixing of each of the first to ninth drive wires (W11 to W33) with a corresponding one of the first to ninth coupling portions (21cl1 to 21c33) is unlocked is referred to as a second state.

Interlocking with the movement of the operating portion 400, the state is switched between the first state and the second state. In other words, interlocking with the movement of the operating portion 400 between the detachment position and the fixed position, the state is switched between the first state and the second state.

The internal gear 29 is configured to interlock with the operating portion 400. In the present embodiment, the joint 28 functions as a transmission member for interlocking the operating portion 400 with the internal gear 29. The internal gear 29 and the joint 28 function as an interlocking portion that interlocks with the operating portion 400 such that the state switches between the first state and the second state interlocking with the movement of the operating portion 400.

Specifically, the internal gear 29 and the joint 28 move part (cam 21cc) of the first rotary member 21cp toward the held portion Wa interlocking with the movement of the operating portion 400 in a state where the catheter unit 100 is attached to the base unit 200. When the cam 21cc moves, the coupling portion 21c is switched between the locked state and the unlocked state.

Alternatively, the internal gear 29 may be configured to be directly moved by the operating portion 400. In this case, the internal gear 29 has the function of an interlocking portion.

### <When Held Portion Wa has Variations in Position in Z Direction>

In the present embodiment, when the catheter unit 100 is attached to the base unit 200 and the operating portion 400 is operated, the state is switched between the fixed state and the unlocked state. At this time, in the unlocked state shown in FIG. 11, a held portion recess center Wd of the held portion Wa and a coupling base protrusion center 21cj are at positions substantially coincide with each other in the negative Y direction. However, the held portion recess center Wd and the coupling base protrusion center 21cj may not coincide with each other due to individual differences that are variations in manufacturing, that is, variations in position due to the dimensions and assembly of components. The operation at this time will be described in detail.

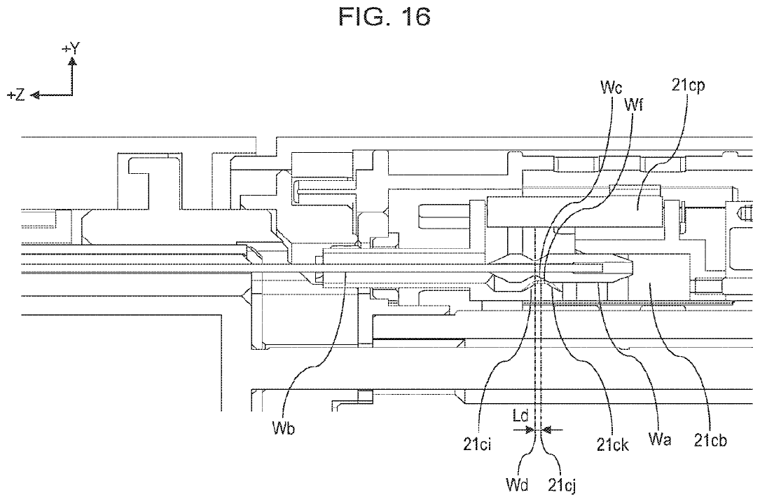

A configuration for fixing the bend drive portion 13 to the coupling device 21 and a configuration for unlocking fixing of the bend drive portion 13 with the coupling device 21 will be described with reference to FIGS. 16, 17, and 18.

FIG. 16 is a sectional view that shows a state of the held portion Wa and the coupling base 21cb in the detachable state.

Although the basic configuration is the same as the configuration described with reference to FIG. 10, the held portion recess center Wd and the coupling base protrusion center 21cj do not coincide with each other in the Z direction in FIG. 16. Specifically, the held portion Wa is at a position shifted by Ld in the positive Z direction from a normal position.

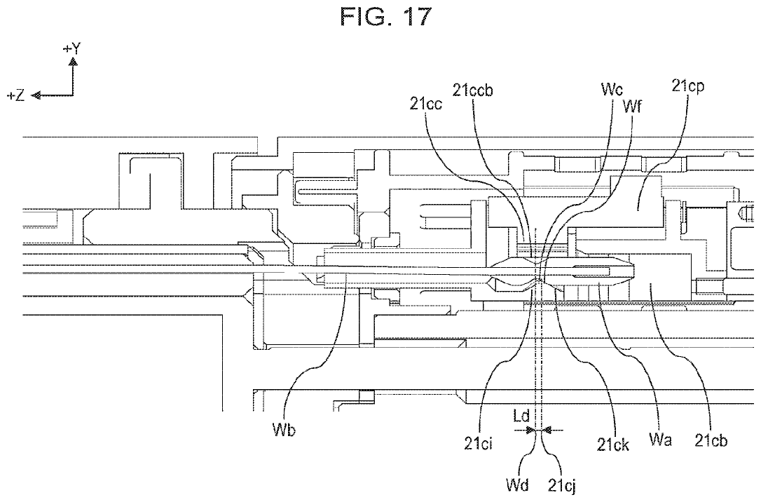

FIG. 17 is a sectional view that shows a state where the held portion Wa and the coupling base 21cb are in process of being engaged with each other.

In FIG. 17, the held portion Wa receives a force from the pressing surface 21ccb of the cam 21cc being rotated and moves in the negative Y direction as described with reference to FIG. 14. At this time, since the held portion recess center Wd and the coupling base protrusion center 21cj do not coincide with each other in the Z direction, a recess portion slope Wf provided on the held portion recess Wc and a protruding portion slope 21ck provided on the coupling base protrusion 21ci contact with each other. Then, due to a force received from the pressing surface 21ccb of the cam 21cc being rotated, the held portion Wa slides obliquely while the recess portion slope Wf and the protruding portion slope 21ck are in contact with each other. At this time, since the coupling base 21cb is fixed, the held portion Wa engages with the coupling base 21cb while moving in the negative Z direction.

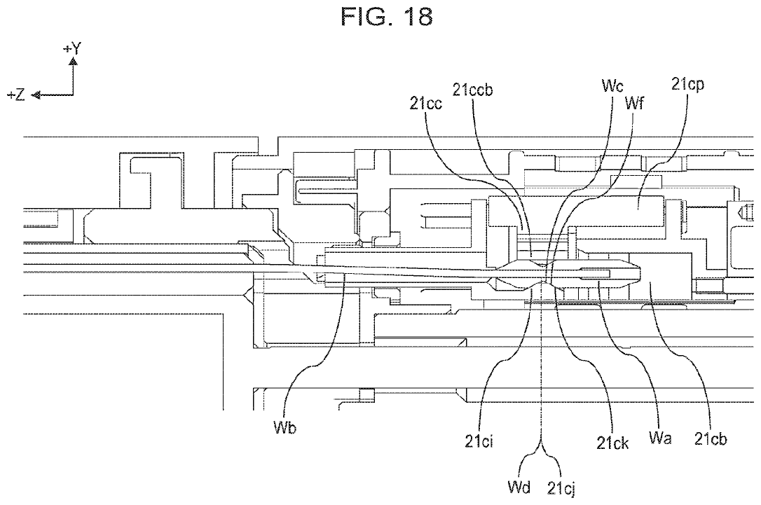

FIG. 18 is a sectional view that shows a state of the held portion Wa and the coupling base 21cb in a state of the fixed position.

FIG. 18, as described with reference to FIG. 15, shows a state where the first rotary member 21cp rotates and is stopped in a state where the stop surface Zb1a provided on the tooth Zb1 contacts with the stopped surface 21cf provided on the coupling base 21cb. At this time, since the held portion Wa is moved in the negative Z direction as described with reference to FIG. 17, the held portion recess center Wd and the coupling base protrusion center 21cj are engaged with each other at positions that coincide with each other in the Z direction. Thus, the held portion Wa is engaged with the coupling base 21cb and fixed in a state of being pressed by the cam 21cc.

Thus, by providing a slope on each of the held portion recess Wc and the coupling base protrusion 21ci, even in a state where the held portion recess center Wd and the coupling base protrusion center 21cj do not coincide with each other in the Z direction, the held portion Wa and the coupling base can be engaged with each other and fixed.

The position that the held portion Wa is shifted by Ld in the positive Z direction has been described as an example this time. The above-described advantage is obtained when there is a positional relationship in which the recess portion slope Wf and the protruding portion slope 21ck can contact with each other in the Z direction. The recess portion slope Wf of the held portion Wa is also provided at a position opposite to the held portion recess center Wd, and the protruding portion slope 21ck of the coupling base is also provided at a position opposite to the coupling base protrusion center 21cj. Thus, even when the held portion Wa is shifted not in the positive Z direction but in the negative Z direction, a similar advantage is obtained when there is a positional relationship in which the recess portion slope Wf and the protruding portion slope 21ck on the opposite side can contact with each other.

### <Storage of Catheter not Attached>

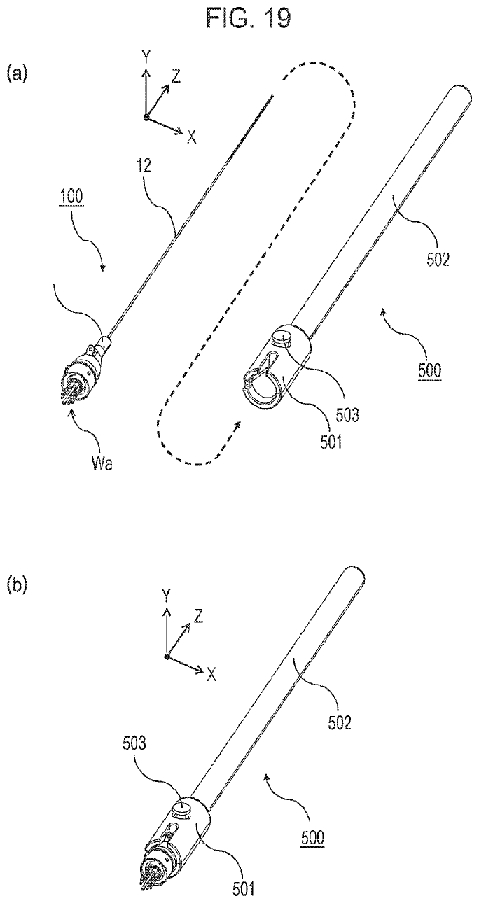

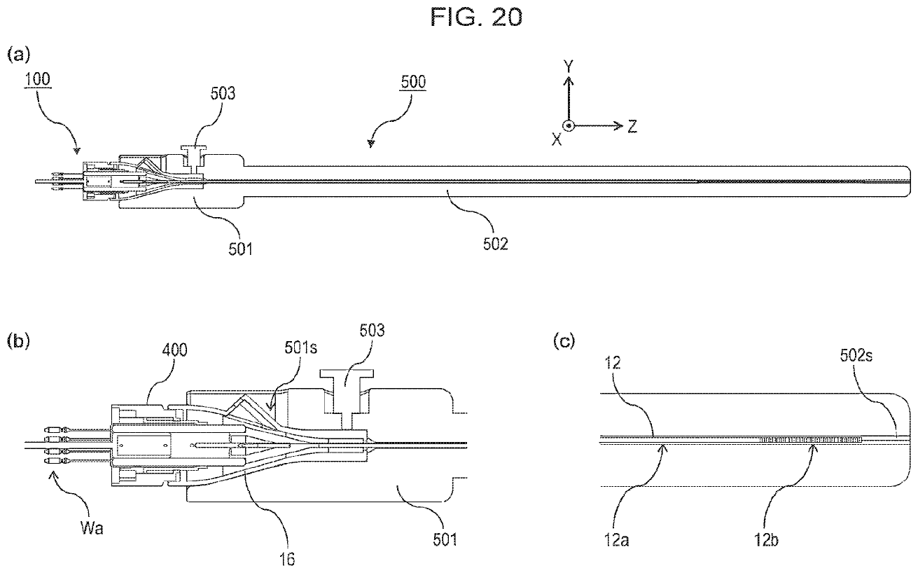

A storage configuration of the catheter unit 100 in a state not attached to the base unit 200 will be described with reference to FIGS. 19 and 20.

The medical device 1 according to the present embodiment is a device capable of freely changing the posture of the bendable portion 12 with the coupling device 21 described with reference to FIG. 6 and the operation configuration of the coupling device 21 described with reference to FIGS. 9 to 15 by, after the drive wire W is coupled to the wire drive portion 300, controlling the plurality of driving sources M independently of one another and moving the drive wire W in the extending direction of the catheter 11.

On the other hand, in a state where the catheter unit 100 is not attached to the base unit 200, the motion of the drive wire W in the extending direction of the catheter 11 is not restricted, so the drive wire W and the held portion Wa are configured to be movable when the posture of the bendable portion 12 is changed. Therefore, after completion of assembly of the catheter unit 100, it is desirable that a situation in which the bendable portion 12 deforms due to the action of some external force before being attached to the base unit 200 and the held portion Wa is coupled to the coupling portion 21c at an unintended position does not occur.

For this reason, as shown in FIG. 19, the catheter unit 100 according to the present embodiment is configured to allow attachment of a catheter case 500 that prevents application of an external force to the catheter 11 after completion of assembly in a manufacturing process and before the catheter unit 100 is attached to the base unit 200 by a user.

FIG. 19(a) is an outer appearance view before the catheter unit 100 and the catheter case 500 are attached to each other. FIG. 19(b) is an outer appearance view after attachment. For the sake of illustration of the positional relationship among the plurality of held portions Wa of the catheter unit 100 (described later), the wire cover 14 that covers the held portion Wa is omitted from FIG. 19 and the following figures. In the drawings from FIG. 19, the extending direction of the catheter 11 is shown as a Z direction. Directions orthogonal to the Z direction and orthogonal to each other are shown as an X direction and a Y direction.

FIG. 20(a) is a sectional view perpendicular to the X direction in the state shown in FIG. 19(b). FIG. 20(b) is an enlarged view of a proximal end-side cross section in FIG. 20(a). FIG. 20(c) is an enlarged view of a distal end-side cross section.

As shown in FIG. 19(a), the catheter case 500 includes a case base 501 of which the posture is determined when attached to the catheter unit 100 and that is fixed to the catheter unit 100, a catheter accommodation portion 502 that accommodates the catheter 11 in a state where the posture of the bendable portion 12 is straight in the extending direction of the catheter 11, and a case fixing device 503 made up of a screw for fixing the case base 501 to the proximal end cover 16 of the catheter unit 100.

As shown in FIG. 20(b), the case base 501 has a cover accommodation region 501s in which the proximal end cover 16 of the catheter unit 100 can be accommodated. As shown in FIG. 20(c), the catheter accommodation portion 502 has a catheter insertion hole 502s in which the bendable portion 12 of the catheter 11 can be accommodated.

When the catheter unit 100 is inserted into the catheter case 500 to a predetermined position in the direction of an arrow indicated by the dashed line in FIG. 19, that is, in the Z direction, and then the case fixing device 503 is operated, the case base 501 is fixed to the proximal end cover 16 as shown in FIG. 20(b), and attachment completes.

A case fixing portion 501a has such a configuration that the case base 501 is fixed to the proximal end cover 16 by a screw in the present embodiment; however, the configuration is not limited thereto. The case fixing portion 501a may have such a configuration that a fixing device using an elastic engagement portion, such as a magnet and a snap fit, is provided and movement of the case base 501 in the Z direction is restricted after attachment. Attaching/detaching work can be simple.

As shown in FIG. 20(c), the catheter insertion hole 502s is configured to cover the outer shape of the bendable portion 12 as a result of attachment of the catheter case 500 such that the intermediate region 12a and the bendable region 12b of the bendable portion 12 maintain a linear posture without displacement. Deformation of the bendable region 12b is restricted when the bendable portion 12 is accommodated in the catheter insertion hole 502s, and deflection of the intermediate region 12b that is easy to deflect because the intermediate region 12b is mainly made up of the plurality of drive wires W is restricted as a result of fixing of the case base 501 integrated with the catheter accommodation portion 502 to the proximal end cover 16, so the linearity of the overall bendable portion 12 is ensured.

Therefore, movement of the drive wire W is restricted by attaching the catheter case 500, and the position of the held portion Wa in the Z direction is maintained at a preset optimal position at the time when the catheter unit 100 is attached to the base unit 200. In the medical device 1 according to the present embodiment, component parts of the base unit 200-side coupling device 21, corresponding to the plurality of drive wires W that make up the catheter 11, are common, so, when the catheter unit 100 is attached to the base unit 200, a state where the Z directions of the plurality of held portions Wa are aligned as shown in FIG. 20(b).

In addition, the catheter case 500 is configured to be placed in a region where the catheter case 500 does not interfere with attachment to the base unit 200 in a state attached to the catheter unit 100. Specifically, as shown in FIG. 20(b), the operating portion 400 is configured to be exposed to outside even in a state where the catheter case 500 is attached to the catheter unit 100. Therefore, the catheter unit 100 can be attached to the base unit 200 in the process described with reference to mainly FIG. 9 while the catheter case 500 remains attached to the catheter unit 100. With the above-described configuration, unintended movement of the held portion Wa is reliably prevented during work for attaching the units. In other words, the catheter unit 100 is easily attached to the base unit.

In the present embodiment, the configuration in which the catheter case 500 has a hole and a recess portion in which the catheter unit 100 can be inserted in the Z direction has been described; however, the configuration is not limited thereto. The catheter case 500 may have such a case configuration that the catheter case 500 is made up of two members divided by a plane passing through the center of a cylindrical shape of the catheter insertion hole 502s, the catheter unit 100 is accommodated so as to be adapted to a groove shape of one of the members, which has the cover accommodation region 501s and the catheter insertion hole 502s, and the other one of the members is assembled to cover. Work for attaching the catheter case 500 can be simple.

### Second Embodiment

Next, a second embodiment (second embodiment) of the present disclosure will be described. Hereinafter, elements having substantially the same configuration and operation to those of the first embodiment are not shown in the drawings or will be described by using like reference signs assigned.

In the first embodiment, the configuration in which the unit is inserted and accommodated in the catheter case 500 in a state where the catheter 11 at the completion of manufacture of the catheter unit 100 remains in a linear posture state has been described. In the present embodiment, a storage configuration in which the catheter 11 is accommodated without maintaining the linear posture of the catheter 11 will be described.

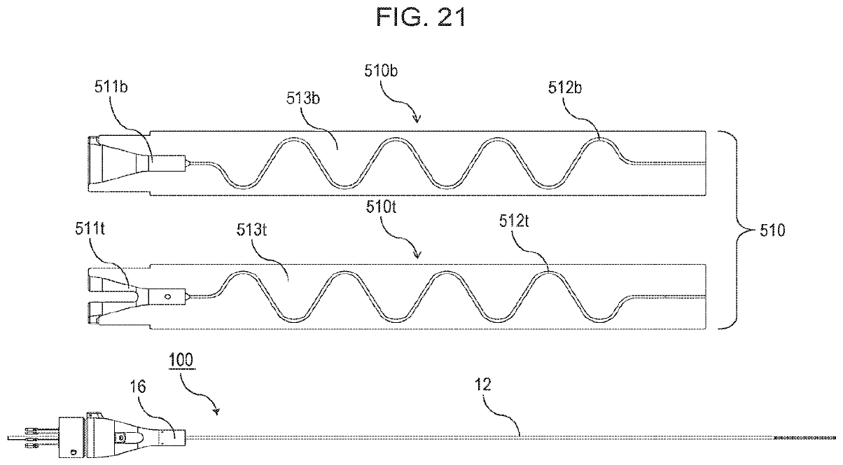

FIG. 21 is an exploded view for illustrating the structure of a catheter case 510 according to the present embodiment and an outer shape view of the catheter unit 100 in a state where the catheter case 510 is not attached. A configuration different from that of the first embodiment is that, in the catheter case 510, a catheter accommodation recessed groove that accommodates the bendable portion 12 is made up of two case members 510b, 510t formed non-linearly and symmetrical with each other.

More specifically, a cover accommodation recessed groove 511b and a catheter accommodation recessed groove 512b are formed on a surface 513b of the case member 510b, and a cover accommodation recessed groove 511t and a catheter accommodation recessed groove 512t are formed on a surface 513t of the case member 510t. The case member 510b and the case member 510t are combined such that the surface 513b and the surface 513t are mated with each other to form the catheter case 510. At this time, the catheter accommodation recessed groove 512b and the catheter accommodation recessed groove 512t form a symmetrical shape with respect to the mating surface of the case members 510b, 510t, and forms a space in which the bendable portion 12 can be accommodated.

As shown in FIG. 21, the accommodation shape of the bendable portion 12 of the catheter 11 according to the present embodiment is such that, in the first embodiment, the accommodation portion for the intermediate region 12b other than the proximal end and distal end of the bendable portion 12 is formed in a wavy curve and not in a linear shape.

As steps of attaching the catheter case 510 to the catheter unit 100, the proximal end cover 16 of the catheter unit 100 is accommodated in the cover accommodation recessed groove 511b, and, in order from the proximal side of the bendable portion 12, the bendable portion 12 is accommodated into the catheter accommodation recessed groove 512b while being elastically deformed.

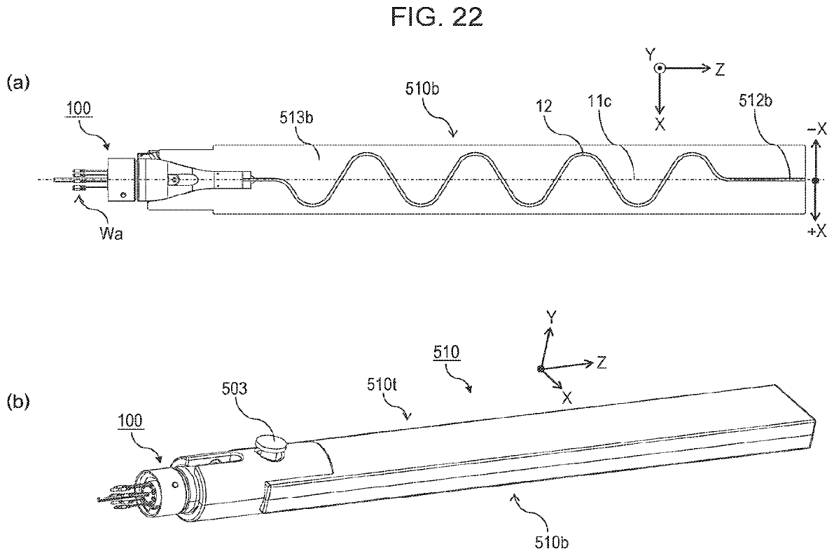

FIG. 22(a) shows a state where the catheter unit 100 is accommodated in the case member 510b. In this state, for the case member 510t, the surface 513t is mated with the surface 513b such that the bendable portion 12 is accommodated in the catheter accommodation recessed groove 512t, and the case members 510b, 510t are fixed by a device, such as a screw.

FIG. 22(b) is an outer appearance view that shows a state where attachment of the case member 510t is complete from the state shown in FIG. 22(a). Lastly, as in the case of the first embodiment, the case fixing device 503 provided for the catheter case 510 is operated to fix the catheter case 510 to the catheter unit 100, and attachment work completes.

As described above, when the accommodation portion for the bendable portion 12 of the catheter 11 is configured in a wavy curve, the overall length of the catheter 11 in the extending direction (Z direction) in a state where the catheter case 510 is attached is shortened as compared to the state of the catheter unit 100 alone, so the size of the packing state during shipping is reduced.

In the present embodiment, the wavy shape of the accommodation portion for the bendable portion 12 is configured to draw a sine curve periodical pattern with a reference line set to a central axis 11c of the catheter 11 in a linear shape before accommodation except for the proximal end and distal end of the bendable portion 12, and the wavy shape is provided such that the total length of the recessed groove in each of a positive X-side region and a negative X-side region with respect to the central axis 11c is equal.

With this configuration, an arc length difference between the inner and outer drive wires W, which occurs when the bendable portion 12 is bent, is cancelled out between the positive X-side region and the negative X-side region, and the positions of the held portions Wa in the Z direction at the proximal ends of the plurality of drive wires W can be brought into coincidence with one another. Therefore, the catheter unit 100 can be attached to the base unit 200 in the process similar to that of the first embodiment.

In the present embodiment, the wavy shape of the accommodation portion for the bendable portion 12 is a sine curve; however, the configuration is not limited thereto. Without regard to periodicity, a shape for guiding the drive wire W is provided in the catheter case 510 such that the total length of the drive wire W accommodated in each of the positive X-side region and the negative X-side region with respect to the central axis 11c is equal. Thus, the catheter unit 100 can be similarly held in a state where the positions of the held portions Wa in the Z direction are aligned.

### Third Embodiment

Next, a third embodiment (third embodiment) of the present disclosure will be described. Hereinafter, elements having substantially the same configuration and operation to those of the first embodiment are not shown in the drawings or will be described by using like reference signs assigned.

In the second embodiment, the configuration in which the total length of the catheter accommodated while being bent on each side with respect to the central axis of the catheter 11 in a linear state before accommodation at completion of manufacture of the catheter unit 100 is equal has been described. In the present embodiment, the storage configuration of the catheter 11 of which the bend is gentle and the total length reduces will be described.

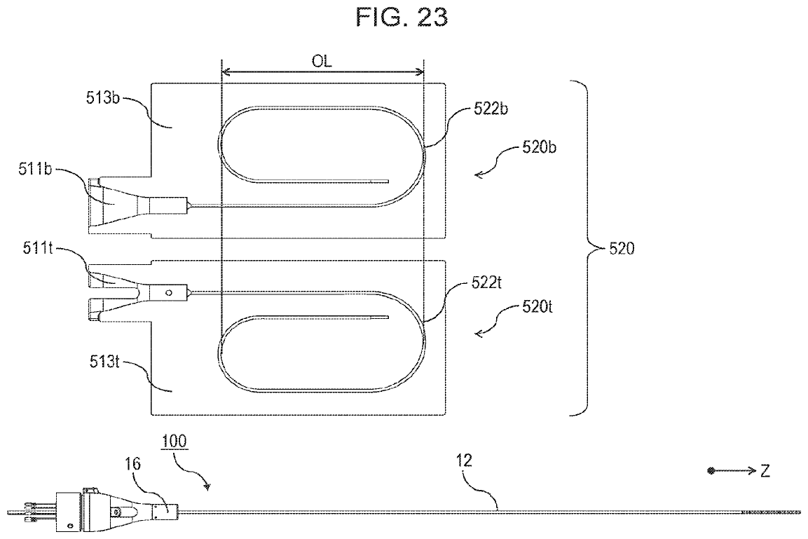

FIG. 23 is an exploded view for illustrating the structure of a catheter case 520 according to the present embodiment and an outer shape view of the catheter unit 100 in a state where the catheter case 510 is not attached. The catheter case 520, as in the case of the second embodiment, is made up of two case members 520b, 520t in which catheter accommodation recessed grooves for accommodating the bendable portion 12 are formed in contrast to each other. On the other hand, a configuration different from that of the second embodiment is that the catheter accommodation recessed grooves are formed in a spiral shape.

More specifically, a cover accommodation recessed groove 511b and a catheter accommodation recessed groove 522b are formed on a surface 513b of the case member 520b, and a cover accommodation recessed groove 511t and a catheter accommodation recessed groove 522t are formed on a surface 513t of the case member 520t. The case member 520b and the case member 520t are combined such that the surface 513b and the surface 513t are mated with each other to form the catheter case 520. At this time, the catheter accommodation recessed groove 522b and the catheter accommodation recessed groove 522t form a symmetrical shape with respect to the mating surface of the case members 520b, 520t, and forms a space in which the bendable portion 12 can be accommodated.

As shown in FIG. 23, the accommodation shape of the bendable portion 12 of the catheter 11 in the present embodiment is, as compared to the second embodiment, not a shape that extends while drawing a curvature in the Z direction but is formed in a spiral shape such that the accommodation portion for the bendable portion 12 extends toward the inner side with respect to the proximal end of the catheter 11.

When the accommodation shape in which the bendable portion 12 is bent, described in the second embodiment, is implemented, constraints on the curvature of bending occur depending on the material and shape of a component so that the component that makes up the catheter 11 does not buckle or plastically deform, so the material and structure of the catheter 11 influences the overall length of the catheter case 510 in the Z direction. On the other hand, by forming the accommodation shape of the bendable portion 12 in a spiral shape as in the case of the present embodiment, the accommodation shapes of the plurality of bendable portions 12 are formed in an OL region in the Z direction, with the result that the overall length of the catheter case 520 in the Z direction can be further shortened.

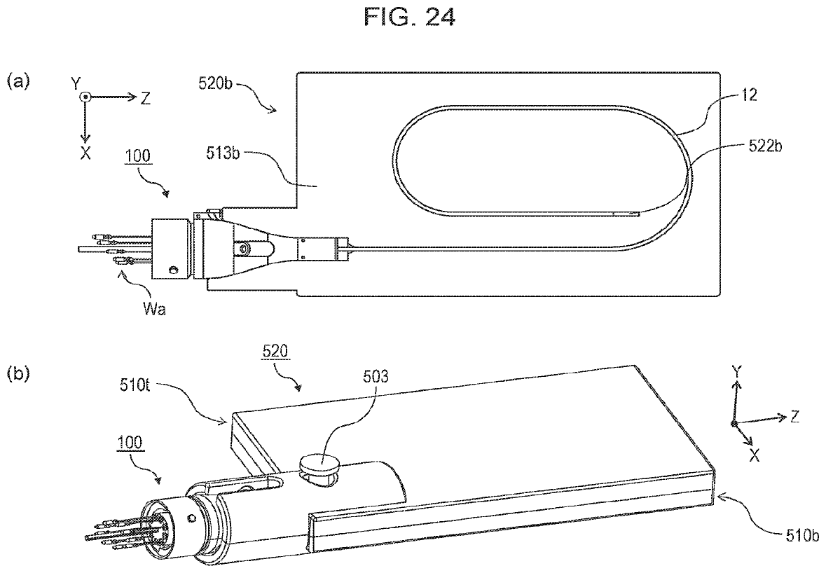

FIG. 24(a) shows a state where the catheter unit 100 is accommodated in the case member 520b. In this state, for the case member 520t, the surface 513t is mated with the surface 513b such that the bendable portion 12 is accommodated in the catheter accommodation recessed groove 522t, and the case members 520b, 520t are fixed by a device, such as a screw.

FIG. 24(b) is an outer appearance view that shows a state where attachment of the case member 520t is complete from the state shown in FIG. 24(a). Lastly, as in the case of the first embodiment and the second embodiment, the case fixing device 503 provided for the catheter case 510 is operated to fix the catheter case 520 to the catheter unit 100, and attachment work completes.

As described above, in the first embodiment and the second embodiment, since the accommodation shape of the catheter 11 is configured symmetrical with respect to the central axis 11c (see FIG. 21), the positions of the held portions Wa of the drive wires W in the Z direction coincide with one another in a state where the catheter case 500 or the catheter case 510 is attached; whereas, the accommodation shape of the catheter 11 according to the present embodiment is an asymmetric shape as shown in FIG. 23, so the positions of the held portions Wa in the Z direction do not coincide with one another.

Specifically, as for the plurality of drive wires W that make up the inside part of the bendable portion 12, there occurs an arc length difference between the drive wires passing on the outer side of the spiral accommodation shape and the drive wires passing on the inner side, so the positions of the corresponding held portions Wa move while shifting by the amount of arc length difference during storage as compared to the positions during non-storage. The arc length difference is a value that can be learned in advance depending on the setting of the shape of each of the catheter accommodation recessed grooves 522b, 522t.

Therefore, when the catheter unit 100 to which the catheter case 520 as described in the present embodiment is attached is attached to the base unit 200, the wire drive portion 300 described with reference to FIG. 6 can be driven by the controller 3 to move the coupling device 21 corresponding to each of the drive wires W by a distance of a predetermined value in advance. With such a configuration, even with the catheter case 520 having the asymmetric accommodation shape of the catheter 11, reliable attachment of the units is achieved while unintended movement of the held portions Wa before attachment is prevented.

### REFERENCE SIGNS LIST

100: catheter unit
200: base unit
21: coupling device
21c: coupling portion
400: operating portion
500: catheter case
W: drive wire
Wa: held portion

1. A catheter kit comprising:
a catheter unit including
a linear member,
a frame guiding the linear member,
a first bendable portion that bends when a first linear member of the linear member is driven, and
an intermediate portion that supports between the first bendable portion and the frame; and
a catheter case provided in an accommodation space accommodating the catheter unit, wherein
a restriction portion is provided in the accommodation space, the restriction portion is used to, when the catheter unit and a base unit including a driving source that drives the linear member are coupled to each other, calibrate a coupled state between the linear member and the driving source.

2. The catheter kit according to claim 1, wherein a plurality of the first linear members that are in correspondence with the first bendable portion each have a first length substantially equal to each other.

3. The catheter kit according to claim 2, wherein
between the first bendable portion and the intermediate portion,
a second bendable portion that bends when a second linear member of the linear member is driven is further provided, and
a plurality of the second linear members that are in correspondence with the second bendable portion each have a second length substantially equal to each other.

4. The catheter kit according to claim 1, wherein a position of an engaged portion of each of the plurality of linear members and a position of a coupling portion of the base unit having the driving source that drives the linear member are configured to coincide with each other.

5. The catheter kit according to claim 1, wherein the first bendable portion of the catheter unit is corrected into a linear shape.

6. The catheter kit according to claim 1, wherein the bendable unit correction case is openable and closable.

7. The catheter kit according to claim 1, wherein the bendable unit correction case also serves as a packing box or a shipping box.

8. A catheter case comprising:
an accommodation space accommodating a catheter unit including
a linear member,
a frame guiding the linear member,
a first bendable portion that bends when the linear member is driven, and
an intermediate portion that supports between the first bendable portion and the frame, wherein
a restriction portion is provided in the accommodation space, the restriction portion is used to, when the catheter unit and a base unit including a driving source that drives the linear member arc coupled to each other, calibrate a coupled state between the linear member and the driving source.
